(19) United States
(12) Patent Application Publication
(10) Pub. No.: US 2011/0021583 A1
(43) Pub. Date: Jan. 27, 2011

(54) ENHANCED TRANSMUCOSAL COMPOSITION AND DOSAGE FORM

(75) Inventors: Richard J. Holl, Park City, UT (US); Matthew Lentz, Salt Lake City, UT (US)

Correspondence Address: Ross J. Oehler;CEPHALON, Inc., 41 MOORES ROAD, PO BOX 4011, FRAZER, PA, 19355, US

(73) Assignee: CEPHALON, INC., Frazer, PA (US)

(21) Appl. No.: 12/860,978

(22) Filed: Aug. 23, 2010

Related U.S. Application Data
(63) Continuation of application No. PCT/US2009/001626, filed on Mar. 13, 2009.
(60) Provisional application No. 61/069,414, filed on Mar. 14, 2008.

(51) Int. Cl. A61K 31/422; A61K 31/4045; A61P 25/06

(52) U.S. Cl. 514/376; 514/415

(57) ABSTRACT

The invention provides a transmucosal pharmaceutical composition comprising an active compound, a bile salt and an osmolality adjusting ingredient, wherein the osmolality adjusting ingredient generates a localized hyperosmotic environment and maintains an osmolality level for a period of time sufficient to produce hypertonicity-facilitated transmucosal transport of said active compound across the mucosal tissue. The invention also provides a solid transmucosal dosage form containing the composition, as well as a method of treatment comprising administering the same. In one embodiment, the active compound is a triptan compound, e.g., sumatriptan and zolmitriptan.

### CROSS REFERENCE RELATED TO APPLICATIONS

This application is a continuation to International Application No. PCT/U.S.09/001,626, filed Mar. 13, 2009, which claims priority to U.S. Provisional Application No. 61/069,414, filed Mar. 14, 2008 (expired), the disclosure of which is incorporated herein by reference in their entireties.

### BACKGROUND

Transmucosal delivery routes, including oral transmucosal delivery, of pharmaceutically active compounds is well known. The advantages associated with transmucosal delivery are also known, which include more direct transport of the active ingredient into the recipient's system while avoiding gastrointestinal interactions and avoiding first pass metabolism. The challenges and difficulties experienced in the pharmaceutical field with transmucosal delivery routes have often been associated with formulating excipient ingredients that are storage stable and rapidly deliver a therapeutically effective dose of the active ingredient into the recipient's system. Even more challenging is the accomplishment of a solid dosage form, e.g., tablet that achieves these objectives.

One such transmucosal formulation technology, known under the trade name ORAVESCENT® (available from CIMA LABS INC. Eden Prairie, Minn.) and described in U.S. Pat. No. 6,200,604, utilizes a combination of effervescent ingredients and pH adjusting substance in a rapidly disintegrating solid oral dosage form (tablet) to enhance the delivery and transport of certain active ingredients across the mucosal tissue.

Various mucosal absorption enhancers, such as sodium taurocholate, are known to facilitate absorption of certain drugs. Other examples of substances known to enhance either dermal or mucosal absorption include terpenes, terpenoids, essential oils, pyrrolidones, fatty acids and esters, sulfoxides, alcohols, glycols, glycerides, phospholipids, cyclodextrins, chelating agents, amino acid derivatives, lipid synthesis inhibitors, enzymes, and the like.

One example of a therapeutic treatment that can benefit from the advantages associated with transmucosal dosage forms is alleviating headaches—specifically migraine headaches. Migraine headaches afflict a significant portion of the population, and symptoms include debilitating pain. Current treatments for migraines include administration of vasoconstrictors, analgesics, and sometimes analgesics in combination with anti-emetics.

Triptan compounds are indole derivatives that are generally known for the treatment of migraine headaches. Pharmaceutically useful triptan salts include rizatriptan benzoate, naratriptan hydrochloride, frovatriptan succinate, eletriptan hydrobromide and almotriptan malate, and the like. Sumatriptan, or 3-[2-(dimethylamino)ethyl]-N-methyl-1H-indole-5-methane sulfonamide, and its succinate salt, is known to be particularly useful in the treatment of migraine headaches. Sumatriptan and its derivatives are described in U.S. Pat. No. 4,816,470 and U.S. Pat. No. 5,037,845, for example, incorporated herein by reference. Amorphous forms of sumatriptan succinate, and their preparation, are described in U.S. Pat. No. 7,034,162. Sumatriptan succinate has the following chemical structure:

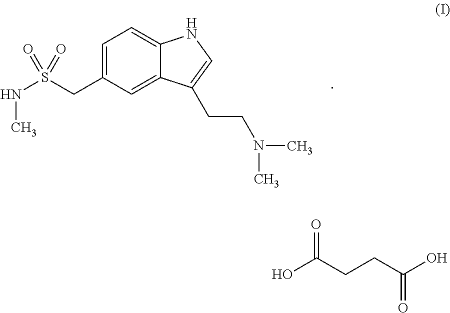

Certain bile salts, such as taurocholate salts, are known to enhance absorption of certain compounds through mucosal tissues. One difficulty associated with formulating certain compounds with bile salts, however, has been their tendency to form salt complexes with certain alkaloid compounds and crystallize or precipitate. This results in decreased amounts of solubilized active ingredients in dosage forms employing bile salts. Ionic forms of certain drugs have a tendency to crystallize, which in turn inhibits the ability of the drug to transport across the mucosal tissue and into the circulatory system. Because of their chemical affinity to one another and significant salt complex formation, formulations containing sumatriptan and bile salts which avoid a salt complex between the same have been difficult to accomplish.

Another problem with certain compounds and bile salts, such as taurocholate, is their tendency to cause local mucosal irritation. Thus, it can be difficult to utilize the absorption properties of taurocholate, for example, while at the same time achieving administrative comfort and reduced irritation.

Formulating compositions which facilitate delivery of the active ingredient in amounts effective to provide their associated therapeutic benefit is an area of significant interest in the pharmaceutical field. Improvements and enhancements to transmucosal drug delivery technology are also being investigated on an ongoing basis.

There exists a need in the pharmaceutical field for dosage forms that improve or enhance transport and delivery of effective amounts of active compounds across the recipient's mucosal tissue in a relatively short period of time to afford the recipient with quick onset of therapeutic benefit of the active.

### SUMMARY

The disclosure provides compositions for transmucosal drug delivery and solid oral dosage forms which can effectively and promptly deliver an active ingredient or compound to a recipient in a therapeutically effective amount. It has been discovered that a solid transmucosal dosage form can be prepared that, by virtue of its ingredients, can enhance and facilitate absorption and transport of an active ingredient to a recipient by exploiting osmolality and tonicity phenomena and their relationship to mucosal absorption. More particularly, it has been discovered that the transmucosal absorption of certain active ingredient or compounds, when formulated with a combination of a bile salt and osmolality adjusting ingredient, can be significantly facilitated or enhanced. Thus, it has been discovered that a solid transmucosal dosage form can be formulated to achieve improved or enhanced transmucosal absorption of an active compound by including a bile salt, e.g., sodium taurocholate, and an osmolality adjusting ingredient, e.g., sodium chloride, within the excipient composition.

The present disclosure provides transmucosal pharmaceutical composition comprising: a) an active compound; b) a bile salt; and c) an osmolality adjusting ingredient; wherein the osmolality adjusting ingredient generates a localized hyperosmotic environment and maintains an osmolality level for a period of time sufficient to produce hypertonicity-facilitated transmucosal transport of the active compound across the mucosal tissue. In one embodiment, the active compound comprises a triptan compound selected from the group consisting of sumatriptan and zolmitriptan, including sumatriptan succinate and free-base zolmatriptan.

In another aspect, the disclosure provides a solid transmucosal dosage form comprising: a) an active compound; b) a bile salt; and c) an osmolality adjusting ingredient; wherein the osmolality adjusting ingredient generates a localized hyperosmotic environment and maintains an osmolality level for a period of time sufficient to produce hypertonicity-facilitated transmucosal transport of the active compound across the mucosal tissue. In one embodiment, the dosage form can be an oral transmucosal buccal or sublingual tablet—preferably sublingual tablet.

In yet another aspect, the disclosure provides a method of treating migraine headaches in a recipient comprising administering to the recipient in need of such treatment a therapeutically effective amount of a transmucosal pharmaceutical composition, the composition comprising: a) a triptan compound as the active compound; b) a bile salt; and c) an osmolality adjusting ingredient; wherein the osmolality adjusting ingredient generates a localized hyperosmotic environment and maintains an osmolality level for a period of time sufficient to produce hypertonicity-facilitated transmucosal transport of the active compound across the mucosal tissue. In particular, the method can comprise the steps of: providing to said recipient the dosage form; placing the dosage form in the recipient's oral cavity; and permitting dosage form to reside in situ and disintegrate in situ for a period of time sufficient to permit transmucosal delivery of a therapeutically effective amount of the active compound across the recipient's mucosa. In one embodiment, the triptan compound can be selected from the group consisting of sumatriptan and zolmitriptan, including sumatriptan succinate and free-base zolmatriptan.

In another aspect, the disclosure provides a method of enhancing oral transmucosal absorption of an active compound in a mammal comprising: a) preparing a composition comprising a combination of active compound together with a bile salt; and b) combining the ingredients of step a) with an osmolality adjusting substance; wherein the combination of ingredients of step a) and step b), in any order, creates osmolality levels consistent with hypertonic conditions and facilitate transport of said active compound across mucosa when said ingredients are placed in situ adjacent oral mucosal tissue of a mammal.

Also provided is a transmucosal pharmaceutical composition comprising:

a) an active compound;

b) a bile salt in an amount of from about 5% to about 30% by weight of the composition; and

c) an osmolality adjusting ingredient present in an amount that results in an osmolality of between about 400 mOs/kg to about 2000 mOs/kg when the osmolality of the transmucosal pharmaceutical composition is measured in 1.5 g of water.

In some embodiments, the active compound is present in an amount of from about 5% to about 25% by weight of the composition, or from about 10 to 20% by weight of the composition, or from about 15% to about 25% by weight of the composition.

In some embodiments, the bile salt is present in an amount of from about 5% to about 30% by weight of the composition, or from about 10% to about 20% by weight of the composition.

The osmolality adjusting ingredient can, in some embodiments, be present in an amount that results in an osmolality of between about 800 mOs/kg to about 1000 mOs/kg when measured in 1.5 g of water.

In some embodiments, the active compound is a triptan compound or a salt of a triptan compound. A triptan compound or salt of a triptan compound can be selected from: rizatriptan benzoate, naratriptan hydrochloride, frovatriptan succinate, eletriptan hydrobromide, almotriptan malate, sumatriptan succinate, and zolmitriptan.

In some embodiments, the bile salt can be selected from a taurocholate salt, a glycocholate salt, a glycodeoxycholate salt, a taurodeoxycholate salt, a cholate salt, a taurochenodeoxycholate salt, a tauroursodeoxycholate salt, and combinations thereof. In some embodiments, the bile salt is a sodium salt, e.g., sodium taurocholate.

The osmolality adjusting ingredient can be selected from potassium chloride, calcium chloride, sodium lactate, sodium chloride, dextrose, mannitol, sucrose, trehalose, and phosphate buffered saline, or mixtures thereof. In some embodiments, the osmolality adjusting ingredient is sodium chloride.

A transmucosal pharmaceutical composition can be in solid dosage form.

Also provided is a method of treating or alleviating one or more symptoms associated with a migraine headache in a subject comprising administering to the subject a therapeutically effective amount of a transmucosal pharmaceutical composition comprising:

a) an active compound;

b) a bile salt in an amount of from about 5% to about 30% by weight of the composition; and

c) an osmolality adjusting ingredient present in an amount that results in an osmolality of between about 400 mOs/kg to about 2000 mOs/kg when the osmolality of the transmucosal pharmaceutical composition is measured in 1.5 g of water.

Also provided is a transmucosal pharmaceutical composition comprising:

a) a triptan compound or salt thereof;

b) sodium taurocholate in an amount of from about 10% to about 20% by weight of the composition; and

c) sodium chloride in an amount that results in an osmolality of the composition of between about 800 mOs/kg to about 1000 mOs/kg when measured in 1.5 g of water.

A method of treating or alleviating one or more symptoms associated with a migraine headache in a subject is also provided, comprising administering to the subject a therapeutically effective amount of the transmucosal pharmaceutical composition comprising:

a) a triptan compound or salt thereof;

b) sodium taurocholate in an amount of from about 10% to about 20% by weight of the composition; and

c) sodium chloride in an amount that results in an osmolality of the composition of between about 800 mOs/kg to about 1000 mOs/kg when measured in 1.5 g of water.

These and other aspects and advantages associated with the disclosure will become apparent from the following disclosure.

### BRIEF DESCRIPTION OF THE DRAWINGS

The invention is further illustrated by the following drawings, none of which are intended to be construed as necessarily limiting the invention.

FIG. 1 is a graph of comparative canine in vivo pharmacokinetic data of various formulations containing sumatriptan succinate.

FIG. 2 is a graph of comparative canine in vivo pharmacokinetic data of various formulations containing sumatriptan succinate.

FIG. 3 is a graph of comparative canine in vivo pharmacokinetic data of various powder blend formulations containing sumatriptan succinate, wherein the formulations vary in osmolality.

FIG. 4 is a graph of comparative canine in vivo pharmacokinetic data of various formulations containing base ingredients of sumatriptan succinate, sodium taurocholate and sodium chloride and varying secondary ingredients.

FIGS. 5A and 5B are graphs of comparative canine in vivo pharmacokinetic data of compressed powder tablets containing sumatriptan succinate and varying in sodium taurocholate and osmolality adjusting ingredient amounts.

FIG. 6 is a graph of comparative canine in vivo pharmacokinetic data of formulations containing zolmitriptan in effervescent formulations.

FIG. 7 is a graph of comparative canine in vivo pharmacokinetic data of formulations containing varying amount of zolmitriptan.

FIG. 8 is a graph of comparative canine in vivo pharmacokinetic data of formulations containing zolmitriptan and differing osmolality adjusting ingredients.

### DETAILED DESCRIPTION OF THE INVENTION

As used herein, the phrase “oral transmucosal,” within the context of drug delivery and absorption, is meant to refer to the pre-peristaltic stage of uptake of the drug via one or more of the mucosal tissue types associated with the oral cavity, e.g., sublingual, buccal, gingival, palatal, esophageal regions of oromucosal tissue. More specifically, what is intended by the phrase is that the primary delivery route of the active ingredient occurs through the mucosal tissue of the oral cavity.

As used herein, the term “about” refers to a range of values from ±10% of a specified value, and functional equivalents thereof unless otherwise specifically precluded. For example, the phrase “about 50 mg” includes±10% of 50, or from 45 mg to 55 mg.

As used herein, the term “therapeutically effective amount” is meant to refer to the amount determined to be required to produce the physiological effect intended and associated with the given active ingredient, as measured according to established pharmacokinetic methods and techniques, for the given administration route.

As used herein, the phrase “oral dosage form”, when used in the general sense, includes orally disintegrable/dissolvable tablets, capsules, caplets, gels, creams, films, sprays, and the like. Within the specific context of the instant invention, the oral dosage form of the invention refers to the pharmaceutical composition of the invention as a solid oral dosage form comprising an active compound accompanied by an excipient formulation which facilitates and enhances oral transmucosal absorption of the active ingredient as defined by the invention.

As used herein, the term “substantially”, unless otherwise defined, is meant to refer to a specific property, characteristic or variable that meets the stated criteria in such measure that one skilled in the art would understand that the benefit to be achieved, or the condition or property desired, is met.

The compositions of the invention are discussed herein within a general context of being “formulated for resident placement within a recipient's oral cavity for transmucosal delivery of said active compound across said recipient's oral mucosal tissue.” This phrase, and like phrases made herein, are meant to indicate that by virtue of the collective combination of ingredients, their individual and combined functionalities, and the techniques used to prepare the dosage form, provide a dosage form that affords delivery of the active ingredient across the recipient's mucosal tissue when placed adjacent thereto for a period of time sufficient to permit such transport.

Transmucosal delivery, including oral transmucosal delivery, avoids common disadvantages associated with conventional gastrointestinal delivery. Such disadvantages include activity loss in the gastrointestinal tract, as well as passage through the liver and first pass metabolism.

The terms “osmolality” and “osmolarity” refer to properties of a particular solution in and of itself. Osmolality is the measure of the number of particles present in solution and is independent of particle size or particle weight, and can be measured only by use of a property of the solution that is dependent only on particle concentration, i.e., “colligative properties.” Colligative properties include vapor pressure depression, freezing point depression, boiling point elevation, and osmotic pressure.

The osmole (Osm) is a unit of measure of the number of moles of chemical compound contributing to a solution's osmotic pressure. Osmolarity is a measure of osmoles of solute per liter of solution, whereas osmolality is a measure of osmoles of solute per kilogram of solvent. Osmolality, as measured in units Osm/kg, can be determined using the following equation:

Osm=ΦnC

wherein

Φ is the osmotic coefficient, and accounts for the degree of non-ideality of the solution, or degree of dissociation of the solute.

n is the number of particles into which a molecule dissociates.

C is the molal concentration of the solution.

The term “tonicity”, on the other hand, refers to the property of a solution in relation to a particular membrane. Tonicity can also be defined as the measure of “effective” osmolarity or effective osmolality. Tonicity is equal to the sum of the concentrations of the solutes which have the capacity to exert an osmotic force across a given membrane. The terms “hypertonic”, “hypotonic” and “isotonic” are defined in reference to a cell membrane by comparing the tonicity of the solution with the tonicity within the intracellular environment. Tonicity is less than osmolality, i.e., it equals the osmolality less the concentration of ineffective solutes.

Within the context of the invention, osmolality adjusting ingredient of the composition generates, when combined with a bile salt in the composition, a localized hyperosmotic environment and maintains an osmolality level for a period of time sufficient to produce hypertonicity-facilitated transmucosal transport of said active compound across the mucosal tissue. Put another way, the changes to osmolality, in relation to the composition property, produce changes to tonicity, in relation to the mucosal tissue which when osmolality changes are combined with bile salts, significantly facilitate transport of the active ingredient across the mucosa. It has been discovered that a composition can be formulated to take advantage of this phenomenon for drug delivery.

In general, the invention includes a transmucosal pharmaceutical composition comprising: a) an active compound; b) a bile salt; and c) an osmolality adjusting ingredient. The osmolality adjusting ingredient, in combination with the bile salt in the composition, generates a localized hyperosmotic environment and maintains an osmolality level for a period of time sufficient to produce hypertonicity-facilitated transmucosal transport of the active compound across the mucosal tissue.

The composition of the invention comprises an active compound. More than one active compound or ingredient can be used. In one embodiment, the composition of the invention can comprise an indole compound and its derivatives. Indole compounds include triptan compounds and their salts. Suitable triptan compounds in salt form that can be used in conjunction with the invention can be selected from the group consisting of rizatriptan benzoate, naratriptan hydrochloride, frovatriptan succinate, eletriptan hydrobromide, almotriptan malate, and sumatriptan succinate, and combinations thereof. Some triptans can be delivered in free base form as well, such as zolmitriptan. In one embodiment, the triptan compound can be selected from the group consisting of sumatripan and zolmitriptan, including sumatriptan succinate and free-base zolmatriptan.

The amount of active compound that can be used with the invention can vary according to the desired effective dosage amount and the remaining ingredients in the composition, e.g., dosage form composition ingredients. For sumatriptan succinate as the active ingredient, the amount can generally range from between about 5% by weight and about 25% by weight of the total composition.

It is believed that the composition of the invention may function to deliver other active compounds across the mucosa. As such, it may be possible to use the composition of the invention to deliver other active compounds. Active compounds that it may be possible to deliver using the invention include pharmaceutical ingredients capable of absorption through the mucosa. Pharmaceutically active ingredients include, without limitation, analgesics, anti-inflammatories, antipyretics, antihistamines, antiasthmatics, antidiuretics, antiflatulents, antimigraine agents, antispasmodics, sedatives, antihyperactives, antihypertensives, tranquilizers, decongestants, beta blockers, peptides, proteins, oligonucleotides and other substances of biological origin, and combinations thereof. Other active ingredients are recited in Mantelle U.S. Pat. No. 5,234,957, the text of which is incorporated herein by reference.

The composition of the invention includes at least one bile salt within the composition. This portion of the composition is also hereinafter referred to as the “bile salt component” of the composition. Bile salts as used herein refer to the cationic salt form of its corresponding bile acid, e.g., bile acid taurocholic acid is (sodium) taurocholate as a bile salt. Bile salts that can be used with the invention include, but are not limited to, bile salts selected from the group consisting of: sodium taurocholate (TC), sodium glycocholate (GC), sodium glycodeoxycholate (GDC), sodium taurodeoxycholate (TDC), sodium cholate (C), sodium taurochenodeoxycholate (TCDC), and sodium tauroursodeoxycholate (TUDC), and combinations thereof. Preferably, the bile salt used is the sodium salt of taurocholic acid, i.e., sodium taurocholate. Although for purposes of illustrating the invention sodium is the named cation, it may be possible to use other cations to form bile salts.

The amount of bile salt that can be used will vary according to the particular bile salt selected. Generally, the amount of bile salt will be relatively low, and within a range of from a minimum effective concentration to achieve the benefits of the invention, and an amount corresponding to maximum acceptable toxicity. The minimum and maximum bile salt amount parameters will differ among the various bile salts. For sodium taurocholate, the amount that can be used for the invention when incorporated into a compressed tablet formulation having a target weight of 100 mg, for example, can range from about 5% weight to weight to about 30% weight to weight, preferably between about 10% and about 20% weight to weight of the total composition, or about 10 mg to about 20 mg of a 100 mg tablet.

The composition of the invention further includes an osmolality adjusting ingredient. The osmolality adjusting ingredient generates a localized hyperosmotic environment and maintains an osmolality level for a period of time sufficient to produce hypertonicity-facilitated transmucosal transport of the active compound across the mucosal tissue. Osmolality adjusting ingredients can be ionic or nonionic provided that in use within a composition, they can generate high enough levels of osmolality relative to their weight. Multivalent ionic osmolality ingredients are, however, preferred because they are associated with higher osmolality generation on a per weight basis.

A variety of pharmaceutically acceptable compounds can function as osmolality adjusting ingredients. Suitable osmolality adjusting ingredients that can be used include, but are not limited to, salts, sugars, buffers, electrolytes, tonicifiers, osmotic agents, chelating agents, pore-forming agents, pH modifying agents, disintegrants and antioxidants. Examples of salts that function as osmolality adjusting ingredients include potassium chloride, calcium chloride, sodium lactate and sodium chloride. Examples of sugars that can be used as osmolality adjusting ingredients include dextrose, mannitol, sucrose and trehalose. For dry-state compressed powder dosage forms, sodium chloride is preferred as the osmolality adjusting ingredient. For liquid dosage forms, phosphate buffered saline can be used as the osmolality adjusting ingredient.

The amount of osmolality adjusting ingredient that can be used can vary depending on the particular osmolality adjusting ingredient employed, and provided its functionality (transmucosal delivery facilitation) is achieved without substantially adversely affecting the function of the remaining ingredients. In general, the amount of osmolality adjusting ingredient that can be used is an amount that adjusts the osmolality of the resulting formulation to a range between about 400 mOs/kg to about 2000 mOs/kg, preferably between about 800 mOs/kg up to about 1000 mOs/kg—when measured in 1.5 g of water. Although a general trend has been observed wherein increasing osmolality increased absorption of the active ingredient, the osmolality increase must be balanced in relation to irritation of the mucosal tissue. Thus, it had also been observed that with respect to sodium chloride as the osmolality adjusting agent, osmolality levels greater than about 1000 mOs/kg were accompanied by increased irritation of the mucosal tissue. Optimal sumatriptan absorption occurred in formulations that generate an osmolality condition of approximately 1000 mOs/kg as measured in 1.5 g of water.

It may be possible to use additional absorption-enhancing agents or substances in combination with the pharmaceutical composition of the invention. Examples of absorption-enhancing substances include, but are not limited to, terpenes, terpenoids, essential oils, pyrrolidones, fatty acids and esters, sulfoxides, alcohols, glycols, glycerides, phospholipids, cyclodextrins, chelating agents, amino acid derivatives, lipid synthesis inhibitors, enzymes, and the like.

According to the invention, the absorption enhancement attribute of the pharmaceutical composition should be effective with buccal, sublingual, gingival, palatal, or esophageal mucosal tissues. A preferred administration site of the pharmaceutical composition of the invention can be in the form of an oral transmucosal dosage form for delivery across the buccal or sublingual mucosa. Suitable dosage forms that can be used with the invention include, but are not limited to, buccal, sublingual or gingival tablets. In a preferred embodiment, the dosage form is an oral transmucosal sublingual tablet formulated for resident placement within the recipient's oral cavity adjacent mucosal tissue. At such position, the dosage form resides in situ and disintegrates in situ for a period of time sufficient to permit transmucosal delivery of a therapeutically effective amount of the active compound across the recipient's mucosa.

In a preferred embodiment, the dosage form prepared in accordance with the invention is a compressed solid, oral transmucosal dosage form composed of a monolithic, single phase composition. The dosage form is further preferably robust, higher density, packaging stable, non-porous dosage form having relatively low friability properties and formulated for disintegration in situ upon its placement and residence within the recipient's oral cavity adjacent mucosal tissue and in contact with saliva. Another important aspect of the dosage form prepared according to the invention is that being formulated for transmucosal delivery of the active ingredient(s), the dosage form should not be an ODT or oral disintegrating tablet (i.e., disintegrating within one minute). In order to optimize transmucosal delivery using the invention and achieve desirable systemic absorption of the active, a preferred oral-mucosal residence time for the dosage form is at least 1 minute, preferably ranging from about 5 minutes to about 10 minutes. Generally, oral mucosal residence times can be limited for practical considerations such as patient comfort, acceptance and compliance for drug products.

In the case of orally disintegrating transmucosal tablets as the dosage form, such as a buccal or sublingual oral transmucosal compressed tablet, the dosage form prepared according to the invention can comprise additional ingredients in combination with the pharmaceutical composition of the invention.

An important aspect of the invention is that, in order to fully realize the benefits of a dosage form prepared according to the invention, the residence time of the dosage form adjacent the mucosal tissue must reside in situ for a period of time sufficient: a) to generate a localized hyperosmotic environment; and b) to maintain osmolality conditions at a level producing hypertonicity-facilitated transmucosal transport of the active compound across the mucosal tissue in the presence of bile salt in the dosage form. This time period can vary according to the particular formulation, ingredients and amounts accompanying the composition of the invention. A preferred disintegration time for a compressed tablet dosage form prepared according to the invention can range from between about 5 minutes and 10 minutes upon placement within the recipient's oral cavity adjacent mucosal tissue, e.g., sublingual placement.

A variety of secondary ingredients can accompany the composition of the invention to prepare an associated excipient formulation appropriate for a given active ingredient, provided that such ingredients individually or collectively do not significantly interfere with the osmolality conditions and mechanism essential to the invention. Suitable secondary ingredients include, but are not limited to, fillers, binders, flavoring agents, coloring agents, lubricants, disintegrants, and the like.

The selection of particular secondary ingredients will depend upon the desired properties of the resultant dosage form to be prepared. Nevertheless, it is preferable to exclude ionic polymeric excipient ingredients and bioadhesive polymers, particularly sodium croscarmellose (NaCMC), from compositions prepared according to the invention to reduce or avoid a sequesterant effect on the bile salt component, e.g., sodium taurocholate, thus reducing the effective role of the bile salt on absorption of the active ingredient. Known bile salt sequesterants should be avoided as ingredients within the composition of the invention.

Possible excipients that can be used to prepare a compressed tablet dosage form include potassium phosphate (monobasic) as a buffer; sodium bicarbonate as a disintegrating agent, and/or maltodextrin as a binding agent; microcrystalline cellulose as a filler; magnesium stearate as a lubricant; and sucralose as a sweetener.

According to one embodiment, the composition of the invention can be prepared by methods known and readily available in the pharmaceutical formulating and manufacturing field, e.g., processing and apparatuses for making compressed powder tablets. In general, a blend of ingredients can be initially prepared according to the given formulation. The blend can then be mixed in a suitable tumble mixer (e.g., TURBULA® mixer or Patterson-Kelly V-blender) for a suitable time period. A lubricant, such as magnesium stearate, can be added to the blend and mixed for additional time. Once sufficiently mixed, the blend can then be transferred to a rotary tablet press and compressed into tablets of pre-selected size and weight. The prepared tablets can then be assayed for quality control and packaged using a variety of techniques, such as bulk packaging or blister packaging.

### Method of Treatment

The invention includes a method of treating a disease or disorder comprising administering to a recipient a dosage form containing the pharmaceutical composition of the invention. In one embodiment in the case of migraine headaches as the disease or disorder, the invention provides a method of treating migraine headaches in a recipient comprising administering to the recipient in need of such treatment a therapeutically effective amount of an oral transmucosal pharmaceutical composition of the invention. According to this particular method, the composition comprises: a) a triptan compound as the active compound; b) a bile salt; and c) an osmolality adjusting ingredient; wherein the osmolality adjusting ingredient generates a localized hyperosmotic environment and maintains an osmolality level for a period of time sufficient to produce hypertonicity-facilitated transmucosal transport of the active compound across the mucosal tissue. The term “recipient” is intended to refer to mammals, including humans. Suitable triptan compounds and their salts can be selected from the group consisting of rizatriptan benzoate, naratriptan hydrochloride, frovatriptan succinate, eletriptan hydrobromide, almotriptan malate, and sumatriptan succinate, and combinations thereof. Certain triptans can be used in their free base form, such as zolmitriptan. A preferred method comprises sumatriptan or zolmitriptan as the active ingredient, including sumatriptan succinate and free-base zolmatritpan.

When compressed tablets are prepared as the dosage form according to the invention, the tablets can be placed in situ adjacent mucosal tissue within the recipient's oral cavity in accordance with the desired position intended for its use. Thus, a tablet can be placed under the recipient's tongue for sublingual administration, or between the cheek and gums in the case of a buccally administered tablet. The recipient's own generated saliva initiates the disintegration process of the tablet, which in turn initiates the chemical environment and transport of the active ingredient across the mucosa. Optimal dosage form residence time can vary according to the dosage form size, disintegration time, and ingredients selected for the particular dosage form.

### EXAMPLES

The following examples further illustrate the invention and are not intended to be construed as necessarily limiting the invention. Unless specified otherwise, the anesthetized dog model experiments were conducted under IACUC approval and conducted according to protocol.

### Example 1

### Comparative in vivo Serum Concentration Study

### Anesthetized Canine Model

Anaesthetized dog models were used to evaluate and compare formulations prepared using different ingredients outside of the invention, as well as formulations prepared according to the invention with varying ingredient amounts. Using the anesthetized dog model, samples were placed in test wells in defined surface area and adhered to the buccal mucosa of the canine subjects.

The formulations tested varied in physical form (powder blend and tablet) and adjusted osmolality (between 350 mOs/kg and 1000 mOs/kg via NaCl content), but were consistent in 10 mg sumatriptan dosage amount and 18 mg or 20 mg bile salt (sodium taurocholate). The following comparative formulations were prepared as follows:

Formulation 1 Preparation of Powder Blend Adjusted with Sodium Chloride to 1000 mOs/kg when Measured in 1.5 g Water

Approximately 14 mg of sumatriptan succinate, 18 mg sodium taurocholate and 45.5 mg sodium chloride were measured and deposited into a small test tube. The test tube was capped and the ingredients were blended by manually inverting the test tube several times. Osmolality of the formulation was measured at about 1000 mOs/kg using a WESCOR® vapor pressure osmometer VAPRO® Model 5520 when measured in 1.5 g water. Formulation 1 is summarized as follows:

Formulation 1

| Ingredient: | Amount (mg) | | Amount % wt/wt |
|---|---|---|---|
| Sumatriptan succinate | 14 | mg | 18% |
| Sodium taurocholate | 18 | mg | 23% |
| Sodium chloride | 45.5. | mg | 59% |
| Total: | 77.5 | mg | 100% |

| | |
|---|---|
| Osmolality: | 1000 mOs/kg (referenced to 1.5 g water) |

Formulation 2 Preparation of Powder Blend Adjusted with Sodium Chloride to 350 mOs/kg when Measured in 1.5 g Water

Approximately 14 mg of sumatriptan succinate, 18 mg sodium taurocholate and 45.5 mg sodium chloride were measured and deposited into a small test tube. The test tube was capped and the ingredients were blended by manually inverting the test tube several times. Osmolality of the formulation was measured at about 350 mOs/kg (using a WESCOR®VAPRO® Model 5520) when measured in 1.5 g water.

Formulation 2

| Ingredient: | Amount (mg) | | Amount % wt/wt |
|---|---|---|---|
| Sumatriptan succinate | 14 | mg | 30% |
| Sodium taurocholate | 18 | mg | 39% |
| Sodium chloride | 14.5 | mg | 31% |
| Total: | 46.5 | mg | 100% |

| | |
|---|---|
| Osmolality: | 350 mOs/kg (referenced to 1.5 g water) |

### Formulation 3 Preparation of Compressed Sublingual Bioadhesive Tablet Containing Sodium Taurocholate

A 1 kg powder blend compositions was prepared by dispensing the ingredients on weight/weight basis: 17% polyethylene oxide (POLYOX® WSR N-80 available from Dow Chemical Co., Midland, Mich.), 8.5% sodium carboxymethyl cellulose, 15% maltodextrin (MALTRIN® M150 available from Grain Processing Corp., Muscatine, Iowa), 15% mannitol, 10% potassium phosphate monobasic, 1% magnesium stearate, 0.60% Neotame (available from NutraSweet Co. Chicago, Ill.), 0.75% sucralose, 18.2% sodium taurocholate, and 14% sumatriptan succinate. The maltodextrin, mannitol, potassium phosphate monobasic, neotame, sucralose, sodium taurocholate and sumatriptan succinate were transferred to a suitable container and blended using a low-shear tumble mixer (TURBULA® T10-B from Glenn Mills, Inc., Clifton, N.J.) for a period of 15 minutes at a rate of 15 rpm. Polyethylene oxide and sodium carboxymethyl cellulose were then transferred to the container and blended for an additional 50 minutes at 15 rpm. Magnesium stearate was added and the mixture was blended for an additional 10 minutes at 15 rpm.

The powder blend was then transferred to a rotary tablet press (PICCOLA® Type-D from SMI Inc., Lebanon, N.J.) and compressed into flat-faced, beveled edge, 5/16″ tablets with a target weight of 100 mg. The formulation achieved an osmolality value of about 230 mOs/kg (WESCOR® VAPRO® 5520 from Wescor, Inc., Logan, Utah) when measured in 1.5 g water at compression force of between 4.5 and 5.5 kN.

Formulation 3 had the following ingredients and amounts:

Formulation 3

| Ingredient: | Amount (mg) | | Amount % wt/wt |
|---|---|---|---|
| Sumatriptan succinate | 14.1 | mg | 14% |
| Sodium taurocholate | 18.2 | mg | 18.2% |
| Sodium chloride | n/a | | 0 |
| Polyethylene oxide | 17.0 | mg | 17% |
| Sodium carboxymethylcellulose | 8.5 | mg | 8.5% |
| Maltodextrin | 15.0 | mg | 15% |
| Mannitol | 15.0 | mg | 15% |
| Potassium phosphate monobasic | 10.0 | mg | 10% |
| Magnesium stearate | 1.0 | mg | 1% |
| Neotame | 0.6 | mg | 0.60% |
| Sucralose | 0.75 | mg | 0.75% |
| Total | | | |

| | |
|---|---|
| Osmolality: | 230 mOs/kg (referenced to 1.5 g water) |

Formulation 4 Preparation of Compressed Sublingual Bioadhesive Tablet without Sodium Taurocholate

A 1 kg powder blend compositions was prepared by dispensing the ingredients on weight/weight basis: 22.4% polyethylene oxide (POLYOX® WSR N-80), 11.2% sodium carboxymethyl cellulose, 20% maltodextrin (MALTRIN® M150), 20% mannitol, 10% potassium phosphate monobasic, 1% magnesium stearate, 0.60% neotame, 0.75% sucralose, and 14% sumatriptan succinate. The maltodextrin, mannitol, potassium phosphate monobasic, Neotame, sucralose and sumatriptan succinate were transferred to a suitable container and blended using a low-shear tumble mixer (TUBULA® T10-B) for a period of 15 minutes at a rate of 15 rpm. Polyethylene oxide and sodium carboxymethyl cellulose were then transferred to the container and blended for an additional 50 minutes at 15 rpm. Magnesium stearate was added and the mixture was blended for an additional 10 minutes at 15 rpm.

The powder blend was then transferred to a rotary tablet pres (PICCOLA® Type-D) and compressed into flat-faced, beveled edge, 5/16″ tablets with a target weight of 100 mg and at compression force of between 7.0 and 7.6 kN. The formulation achieved an osmolality value of about 230 mOs/kg (WESCOR® VAPRO® 5520) when measured in 1.5 g water.

Formulation 4 had the following ingredients and amounts:

Formulation 4

| Ingredient: | Amount (mg) | | Amount % wt/wt |
|---|---|---|---|
| Sumatriptan succinate | 14.0 | mg | 14% |
| Sodium taurocholate | 0 | | 0 |
| Sodium chloride | 0 | | 0 |
| Polyethylene oxide | 22.4 | mg | 22.4% |
| Sodium carboxymethyl cellulose | 11.2 | mg | 11.2% |
| Maltodextrin | 20.0 | mg | 20% |
| Mannitol | 20.0 | mg | 20% |
| Potassium phosphate monobasic | 10.0 | mg | 10% |
| Magnesium stearate | 1.0 | mg | 1% |
| Neotame | 0.60 | mg | 0.60% |
| Sucralose | 0.75 | mg | 0.75% |
| Total | 100 | mg | 100% |

| | |
|---|---|
| Osmolality: | 230 mOs/kg (referenced to 1.5 g water) |

Formulation 5 Compressed Tablet Containing Sodium Taurocholate and Sodium Chloride Adjusted to about 1000 mOs/Kg as Measured Using 1.5 g Water

A 100 gram powder blend was prepared by dispensing on a % weight/weight basis the following: 25.7% sodium chloride, 5.0% sodium bicarbonate, 35% potassium phosphate monobasic, 0.6% sucralose, 1.5% magnesium stearate, 18.2% sodium taurocholate, and 14% sumatriptan succinate. Sodium chloride, sucralose, sodium bicarbonate, sumatriptan succinate and sodium taurocholate were transferred into a suitable container and manually blended by inverting the container. Potassium phosphate monobasic was added to the container and blended for an additional 50 minutes at 15 rpm. Magnesium stearate was added to the container and blended for an additional 10 minutes at 15 rpm. The resulting powder blend was then transferred to a rotary tablet press (PICCOLA® Type-D) and compressed into flat-faced beveled edge ¼″ tablets with a target weight of 100 mg (WESCOR® VAPRO® 5520) at a compression force of between 0.5 and 2.0 kN.

Formulation 5 had the following ingredients and amounts:

Formulation 5

| Ingredient: | Amount (mg) | | Amount % wt/wt |
|---|---|---|---|
| Sumatriptan succinate | 14.0 | mg | 14.0% |
| Sodium taurocholate | 18.2 | mg | 18.2% |
| Sodium chloride | 25.7 | mg | 25.7% |
| Sodium bicarbonate | 5.0 | mg | 5.0% |
| Potassium phosphate | 35.0 | mg | 35.0% |
| monobasic | | | |
| Sucralose | 0.6 | mg | 0.6% |
| Magnesium stearate | 1.5 | mg | 1.5% |
| Total | 100 | mg | 100% |

| | |
|---|---|
| Osmolality: | 1000 mOs/kg (referenced to 1.5 g water) |

### In Vivo Canine Experiment

Formulations 1 through 5 were evaluated using anesthetized in vivo canine models (male and female Beagle dogs weighing between 8 kg and 12 kg) and the experiments were conducted under protocol and IACUC approval.

For Formulations 1 and 2, the purpose of the experiments was to evaluate the effects of osmolality on powder compositions containing sumatriptan succinate, sodium taurocholate and sodium chloride on oral transmucosal absorption. Powder samples prepared as Formulations 1 and 2 were introduced to the dogs using 1″ diameter Teflon reservoir positioned and adhered by denture adhesive to the buccal mucosa. The area within the reservoir is hydrated with about 1.5 ml deionized water for approximately 10 minutes. The hydration fluid is aspirated from the reservoir and discarded. The sample formulations are deposited within the reservoir and hydrated with 1.5 ml deionized water. The samples are maintained on site for a period of 60 minutes exposure time, and the sample is aspirated from the test site and retained for analysis. The reservoir is twice rinsed with 1.5 ml deionized water, and the reservoir is removed and the mucosa site cleaned.

Blood samples (3 mL) were collected via indwelling catheter in the cephalic vein at 0, 5, 10, 20, 30, 45, 60, 90, 120 and 150 minute intervals and analyzed using post-dose VACUTAINER® serum separator using mass spectroscopy (Becton-Dickinson, Franklin Lakes, N.J.).

For Formulations 3 and 4, the purpose of the experiments was to evaluate formulations containing bioadhesive within the composition of sumatriptan succinate and sodium taurocholate prepared as solid bioadhesive tablets and the effect on transmucosal absorption. Using a procedure similar to that used for Formulations 1 and 2, the tablets are placed within the reservoir followed by 1.5 mL deionized water. The sample is maintained for a period of about 60 minutes and then the sample is aspirated from the reservoir. 3 mL blood samples were drawn at 0, 5, 10, 20, 25, 30, 40, 55, 75, 90, 120 and 180 minutes, processed using a serum separator, and analyzed using mass spectroscopy.

The study using Formulation 5 was to evaluate solid tablet compositions containing sumatriptan succinate, sodium taurocholate and sodium chloride on transmucosal absorption. Using the similar reservoir procedure and canine models as above, 3 ml samples were drawn at 0, 5, 10, 20, 30, 45, 60, 90, 120 and 150 minute intervals and analyzed.

The following table is a summary of the focal ingredients of the tested formulations 1 through 5 and corresponding adjusted osmolality values:

TABLE 1
Summary of Formulations 1 through 5

| | | | | | Osmolality |
|---|---|---|---|---|---|
| | Sumatriptan | Sodium | Sodium | | (mOs/kg measured |
| Formulation: | succinate | taurocholate | chloride | Other | in 1.5 g H₂O) |
| 1 (powder blend) 77.5 mg | 14 mg | 18 mg | 45.5 mg | | 1000 mOs/kg |
| 2 (powder blend) | 14 mg | 18 mg | 14.5 mg | | 350 mOs/kg |
| 46.5 mg | | | | | |
| 3 (compressed 5/16″ | 14 mg | 18 mg | 0 | | 230 mOs/kg |
| tablet) 100 mg | | | | | |
| 4 (compressed 5/16″ | 14 mg | 0 | 0 | | 230 mOs/kg |
| tablet) 100 mg | | | | | |
| 5 (compressed ¼″ | 14 mg | 18 mg | 25.7 mg | | 1000 mOs/kg |
| tablet) 100 mg | | | | | |

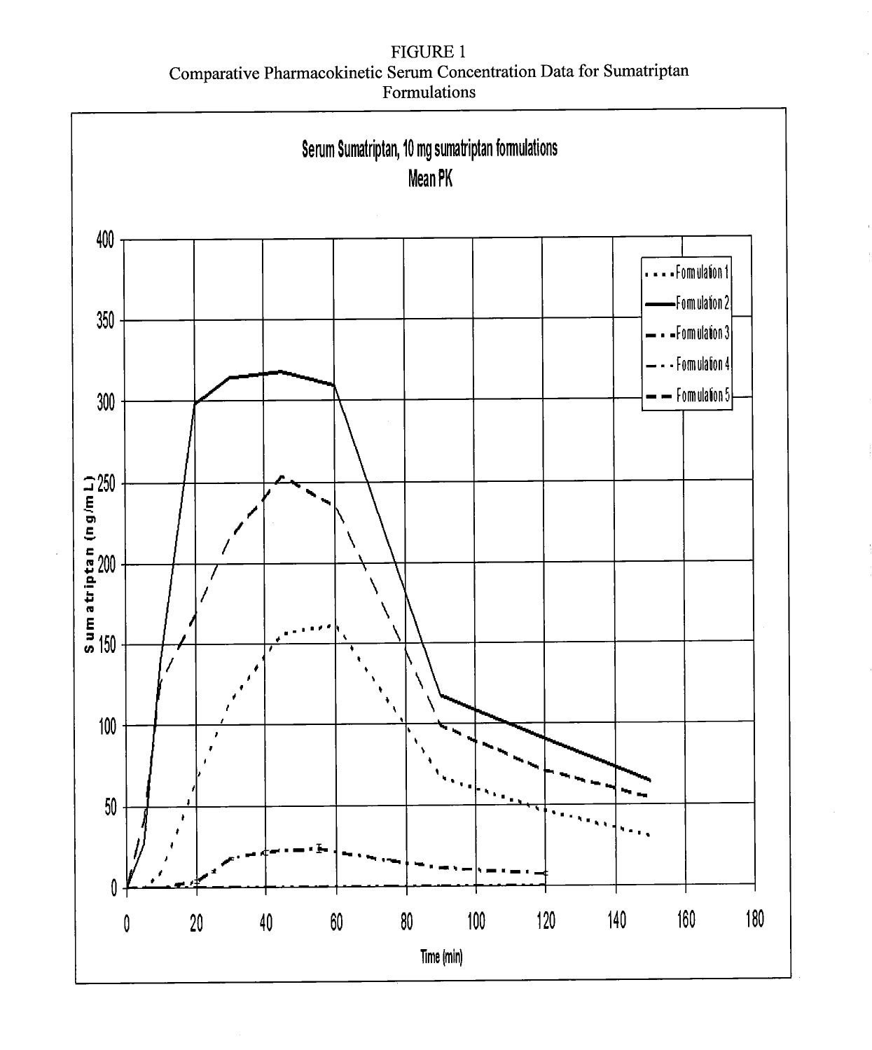

The serum data from Formulations 1 through 5 are shown in FIG. 1. FIG. 1 shows comparative PK Serum Concentration Data for Sumatriptan Formulations using the powder and tablet compositions of Formulations 1 through 5.

As can be seen from the data, the adjustment in osmolality from 350 mOs/kg to 1000 mOs/kg produced a 2-fold increase in sumatriptan absorption as indicated by the serum sumatriptan levels. The data shows that faster and greater sumatriptan transmucosal absorption of sumatriptan can be achieved using formulations containing bile salt (sodium taurocholate) and osmolality adjusting agent (sodium chloride) in concentrations generating hyperosmotic conditions at the delivery site. Further, greater sumatriptan absorption is accomplished using formulations generating higher osmolality values and, correspondingly, greater hyperosmotic conditions.

The results also demonstrate that both powder and compressed powder tablets dosage forms containing sodium taurocholate and sodium chloride were capable of generating osmolality values around 1000 mOs/kg when measured in 1.5 ml of water, and that both produced similar effects on sumatriptan absorption. No serum sumatriptan was detected from the administration of Formulation 4.

### Example 2

### Comparative In Vivo Serum Concentration Study

### Anesthetized Canine Model

In an effort to determine ingredients and conditions that produce little or no sumatriptan absorption, different formulations were prepared and administered to the anesthetized canine models. Various formulations were tested in order to identify ingredients that favorably and adversely impact active transmucosal absorption.

Formulations 1, 2, 3, and 4 were prepared as set forth in Example 1. Formulations 6, 7, 8 and 9 were prepared as follows.

Formulation 6 Preparation of Powder Blend Containing Sumatriptan Succinate, Sodium Taurocholate and Sodium Chloride and Cholestyramine Adjusted to 1000 mOs/Kg when Measured in 1.5 g Water.

Formulation 6 samples were prepared by measuring 14 mg sumatriptan succinate, 18 mg sodium taurocholate, and 45.5 mg sodium chloride and 30 mg cholestyramine into a small test tube. The test tube was capped and the ingredients were blended by manually inverting the test tube under several repetitions. Osmolality for the powdered blend was confirmed at 1000 mOs/kg (WESCOR® VAPRO® 5520) when measured in 1.5 g of water.

Formulation 6

| Ingredient: | Amount (mg) | | Amount (% wt/wt) |
|---|---|---|---|
| Sumatriptan succinate | 14 | mg | 13% |
| Sodium taurocholate | 18 | mg | 17% |
| Sodium chloride | 45.5 | mg | 42% |
| Cholestyramine | 30 | mg | 28% |
| Total | 107.5 | mg | 100% |

| | |
|---|---|
| Osmolality | 1000 mOs/kg (referenced to 1.5 g water) |

### Formulation 7 Preparation of Powder Blend Containing Sodium Taurocholate and Microcrystalline Cellulose

Formulation 7 was prepared by measuring 14 mg sumatriptan succinate, 18 mg sodium taurocholate, microcrystalline cellulose (AVICEL® PH102) into a small test tube. The test tube was capped and the ingredients were blended by manual inversion repeated several times. Osmolality for the powder blends was measured at 100 mOs/kg (WESCOR® VAPRO® 5520) when measured in 1.5 g of water.

Formulation 7

| Ingredient: | Amount (mg) | Amount (% wt/wt) |
|---|---|---|
| Sumatriptan succinate | 14 mg | 23% |
| Sodium taurocholate | 18 mg | 29% |
| Microcrystalline cellulose | 30 mg | 48% |
| Total | 62 mg | 100% |

| | |
|---|---|
| Osmolality | 60 mOs/kg (referenced to 1.5 g water) |

### Formulation 8 Preparation of Powdered Sumatriptan Succinate

Formulation 8 samples were prepared by measuring 14 mg sumatriptan succinate into a small test tube. Osmolality was measured at 30 mOs/kg (WESCOR® VAPRO® 5520) when measured in 1.5 g of water.

Formulation 8

| Ingredient: | Amount (mg) | Amount (% wt/wt) |
|---|---|---|
| Sumatriptan succinate | 14 mg | 100% |

| | |
|---|---|
| Osmolality | 30 mOs/kg (referenced to 1.5 g water) |

### Formulation 9 Preparation of Powder Blend Containing Microcrystalline Cellulose

Formulation 9 samples were prepared by measuring 14 mg sumatriptan succinate and 6 mg microcrystalline cellulose (AVICEL® PH102) into a small test tube. The test tube was capped and the ingredients were blended by manually inverting the tube several times. Osmolality of the composition was 30 mOs/kg using WESCOR® VAPRO® 5520 and measured in 1.5 g of water.

Formulation 9

| Ingredient: | Amount (mg) | Amount % wt/wt |
|---|---|---|
| Sumatriptan succinate | 14 mg | 70% |
| Microcrystalline cellulose | 6 mg | 30% |

| | |
|---|---|
| Osmolality | 20 mOs/kg (referenced to 1.5 g water) |

The focal ingredients used in Formulations 6 through 9 are summarized in the following table:

TABLE 2
Summary of Formulations 6 through 9

| | | | | | Osmolality |
|---|---|---|---|---|---|
| | | | | | (mOs/kg |
| | Sumatriptan | Sodium | Sodium | | measured in |
| Formulation: | succinate | taurocholate | Chloride | Other | 1.5 g H₂O) |
| 6 (powdered | 14 mg | 18 mg | 45.5 mg | 30 mg | 1000 |
| blend) | | | | cholestyramine | |
| 7 (powdered | 14 mg | 18 mg | 0 | 30 mg | 60 |
| blend) | | | | microcrystalline | |
| | | | | cellulose | |
| 8 (powdered | 14 mg | 0 | 0 | 0 | 30 |
| blend) | | | | | |
| 9 (powdered | 14 mg | 0 | 0 | 6 mg | 30 |
| blend) | | | | microcrystalline | |
| | | | | cellulose | |

### In Vivo Canine Experiment

The testing and evaluation of Formulations 1 and 2 were performed according to the experimental conditions and description set forth in Example 1. Likewise, Formulations 3 and 4 were also evaluated as set forth in Example 1.

The purpose of the study with Formulation 6 was to determine the effects on cholestyramine resin in powder compositions containing sumatriptan succinate, sodium taurocholate and sodium chloride on the absorption of sumatriptan succinate through canine buccal mucosa. The study was conducted with anesthetized dog models, and the canine subjects were male and female Beagle dogs weighing between 8 and 12 kg.

Powder samples were introduced to the subjects using a 1″ diameter customized Teflon reservoir adhered to the buccal mucosa using denture adhesive. The buccal mucosa within the reservoir was initially hydrated with 1.5 mL deionized water for a period of about 10 minutes. The hydration fluid is aspirated from the reservoir and discarded. Next, 1.5 mL of deionized water was added into the containers and mixed with the powder compositions to dissolve or suspend the powder samples. The mixture is transferred into the reservoir and maintained in the reservoir for a period of about 60 minutes exposure time. After 60 minutes, the sample is aspirated from the reservoir and retained for subsequent analysis. Then reservoir is then twice rinsed with 1.5 mL deionized water and then discarded along with the reservoir. The site is cleaned from remaining adhesive.

Blood samples (3 mL) were drawn via in-dwelling catheter in the cephalic vein into VACUTAINER® serum separator at time intervals 0, 5, 10, 20, 30, 45, 60, 90, 120 and 150 minutes post-dose. The samples were analyzed for serum sumatriptan content using mass spectroscopy using methods and equipment known in the art.

Formulation 7 was evaluated using procedure similar to that for Formulation 6. Samples (3.0 ml) were taken at time intervals 0, 5, 10, 20, 25, 30, 40, 55, 75 and 90 minutes post-dose using serum separator VACUTAINER®. Samples were analyzed for sumatriptan content using mass spectroscopy.

Formulation 8 was evaluated to determine the effects of high osmolality on sumatriptan absorption on canine buccal mucosa. The procedure was similar to that described above, except that the samples of powdered sumatriptan succinate were added directly into the reservoir followed immediately by 1.5 ml osmolality buffer containing 1.44 g/L KH₂PO₄, 90.0 g/L NaCl, and 7.95 g/L Na₂HPO₄.7H₂O. The osmolality of the buffer was measured by vapor pressure osmometer (WESCOR® VAPRO® 5520) to be 3080 mOs/kg. The sample was maintained within the reservoir for a period of about 60 minutes and removed/rinsed. Blood samples (3 ml) were drawn at times 0, 5, 10, 20, 30, 45, 60, 90, 120 and 180 minutes.

Formulation 9 was tested to evaluate the effects of microcrystalline cellulose in powder compositions containing sumatriptan succinate on transmucosal absorption. The canine model was performed in accordance with the procedures above. The mucosa and reservoir were hydrated with 1.5 mL deionized water for 10 minutes prior, removed, and followed by deposit of the powder blend and subsequent hydration with 1.5 mL deionized water and 60 minutes exposure time. Blood samples (3 mL) were taken at time intervals 0, 5, 10, 20, 25, 30, 40, 55, 75 and 90 minutes.

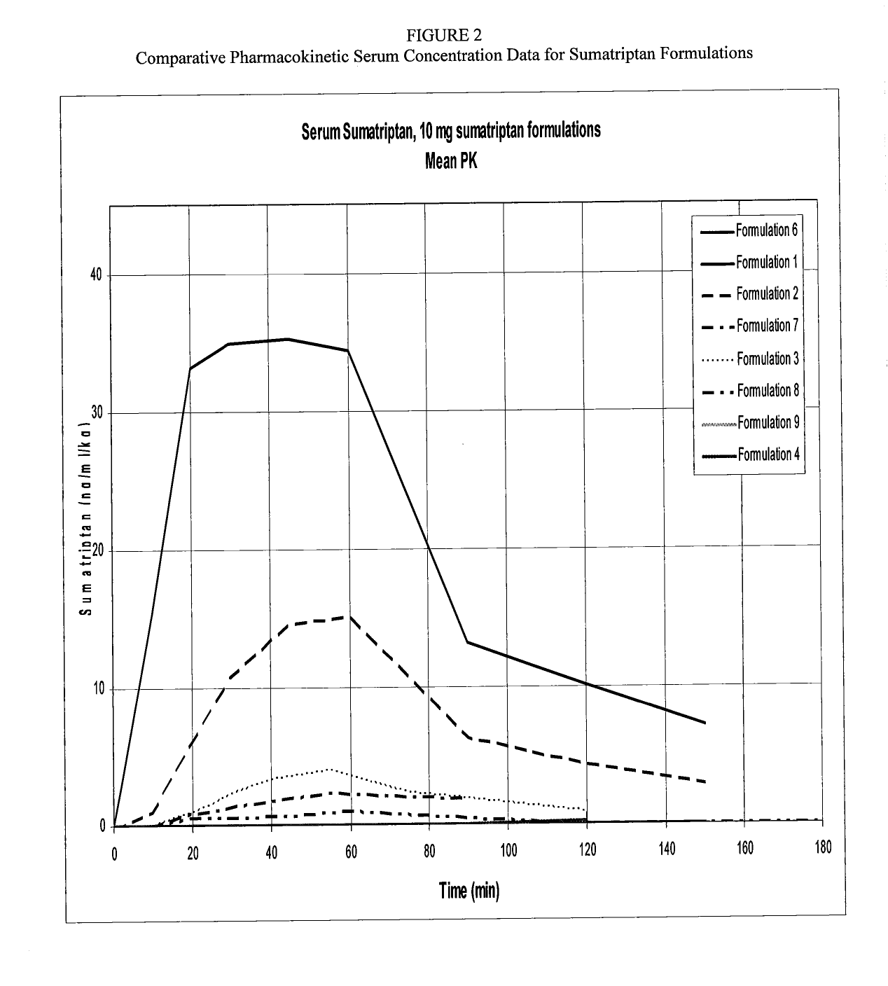

FIG. 2 shows the comparative pharmacokinetic data (serum concentration versus time) of formulations containing sumatriptan succinate in either uncompressed powders or compressed powder tablets and contacted with canine buccal mucosa. Formulations 1 through 4 were prepared and tested as found in Example 1. Formulation 6 was a powder blend containing sumatriptan succinate, sodium taurocholate and sodium chloride and cholestyramine resin—a known bile acid salt sequesterant. Formulation 7 was a powder blend containing sumatriptan succinate and sodium taurocholate without sodium chloride. Formulation 8 contained sumatriptan succinate alone with phosphate buffer solution and sodium chloride. Formulation 9 contained sumatriptan succinate with microcrystalline cellulose.

As can be seen from the data, the results show that serum sumatriptan levels are low or not observed in powder compositions containing only sumatriptan succinate, sumatriptan succinate in combination with only sodium taurocholate, or sumatriptan succinate in combination with only osmolality adjusting ingredients. In other words, it was observed that to achieve higher levels of sumatyriptan absorption through canine buccal mucosa, sumatriptan succinate should be accompanied by a bile salt (sodium taurocholate) and osmolality adjusting ingredients in amounts that generate hyperosmotic conditions. This was even observed with Formulation 8—a formulation generating high osmolality conditions but absent sodium taurocholate.

No serum sumatriptan levels were detected from administration of Formulations 4, 6 and 9. Formulations 4 and 9 do not contain sodium taurocholate, whereas Formulation 6 contained cholestyramine resin. Cholestyramine is a known bile salt sequesterant and based on the data, effectively inhibited sumatriptan absorption under conditions that would otherwise, in the absence of sequesterant, permit elevated absorption of sumatriptan (bile salt plus osmolality adjusting ingredient).

### Example 3

### Comparative Powder Blend Formulations with Various Osmolalities

Powder blend formulations containing 14 mg sumatriptan succinate and 18 mg sodium taurocholate were prepared, each formulation prepared as 1.5 mL contact solutions having varying osmolalities adjusted with sodium chloride as the osmolality agent. Four osmolalities were tested: 350 mOs/kg, 750 mOs/kg, 1000 mOs/kg and 2150 mOs/kg.

Formulation 10 was a powder blend with adjusted osmolality of 750 mOs/kg prepared by initially combining 14 mg sumatriptan succinate, 18 mg sodium taurocholate and 32.3 mg sodium chloride into a small test tube. The tube was capped and manually inverted several times to mix the ingredients. Osmolality of the composition was adjusted to 750 mOs/kg measured by WESCOR® VAPRO® 5520 when measured in 1.5 g water.

Formulation 10

| | | |
|---|---|---|
| Ingredient: | Amount (mg) | Amount (% wt/wt) |

| | | | |
|---|---|---|---|
| Sumatriptan succinate | 14 | mg | 22% |
| Sodium taurocholate | 18 | mg | 28% |
| Sodium chloride | 32.3 | mg | 50% |
| Total: | 64.3 | mg | 100% |

| | |
|---|---|
| Osmolality | 750 mOs/kg (referenced to 1.5 g water) |

For Formulation 11, a powder blend containing 14 mg sumatriptan succinate, 18 mg sodium taurocholate and 97.8 mg sodium chloride was prepared by combining the ingredients into a capped vial and manually inverting several times. Osmolality was adjusted to 2150 mOs/kg measured using WESCOR® VAPRO® 5520 in 1.5 g water.

Formulation 11

| Ingredient: | Amount (mg) | | Amount (% wt/wt) |
|---|---|---|---|
| Sumatriptan succinate | 14 | mg | 11% |
| Sodium taurocholate | 18 | mg | 14% |
| Sodium chloride | 97.8 | mg | 75% |
| Total: | 129.8 | mg | 100% |

| | |
|---|---|
| Osmolality | 2150 mOs/kg (referenced to 1.5 g water) |

The formulations of this experiment are summarized in the following table:

TABLE 3
Summary of Formulations 1, 2, 10 and 11

| | | | | | Osmolality |
|---|---|---|---|---|---|
| | Sumatriptan | Sodium | Sodium | | (mOs/kg measured in |
| Formulation: | succinate | taurocholate | chloride | Other | 1.5 g H₂O) |
| 1 (powder blend) | 14 mg | 18 mg | 45.5 mg | | 1000 mOs/kg |
| 100 mg | | | | | |
| 2 (powder blend) | 14 mg | 18 mg | 14.5 mg | | 350 mOs/kg |
| 100 mg | | | | | |
| 10 (powder blend) | 14 mg | 18 mg | 32.3 mg | | 750 mOs/kg |
| 11 (powder blend) | 14 mg | 18 mg | 97.8 mg | | 2150 mOs/kg |

### In Vivo Canine Experiment

Formulations 1, 2 10 and 11 were evaluated to determine effects of varying osmolality conditions on transmucosal absorption using powder compositions containing primary ingredients sumatriptan succinate, sodium taurocholate and sodium chloride. Using the anesthetized dog model, the solutions were deposited onto the canine mucosa with a contact period of about 60 minutes. The site was initially hydrated with 1.5 mL deionized water for 10 minutes prior to deposition of the composition. The hydration fluid was aspirated from the reservoir and discarded. The compositions were pre-dissolved in 1.5 mL de-ionized water prior to transfer into the reservoir. After residence for 60 minutes, the reservoir was aspirated and the composition rinsed off and discarded.

Blood samples were collected from the canine subjects at time intervals 0, 5, 10, 20, 30, 45, 60, 90, 120 and 150 minutes post-dose serum separator, analyzed, and serum concentrations of sumatriptan calculated. The results were plotted by graph.

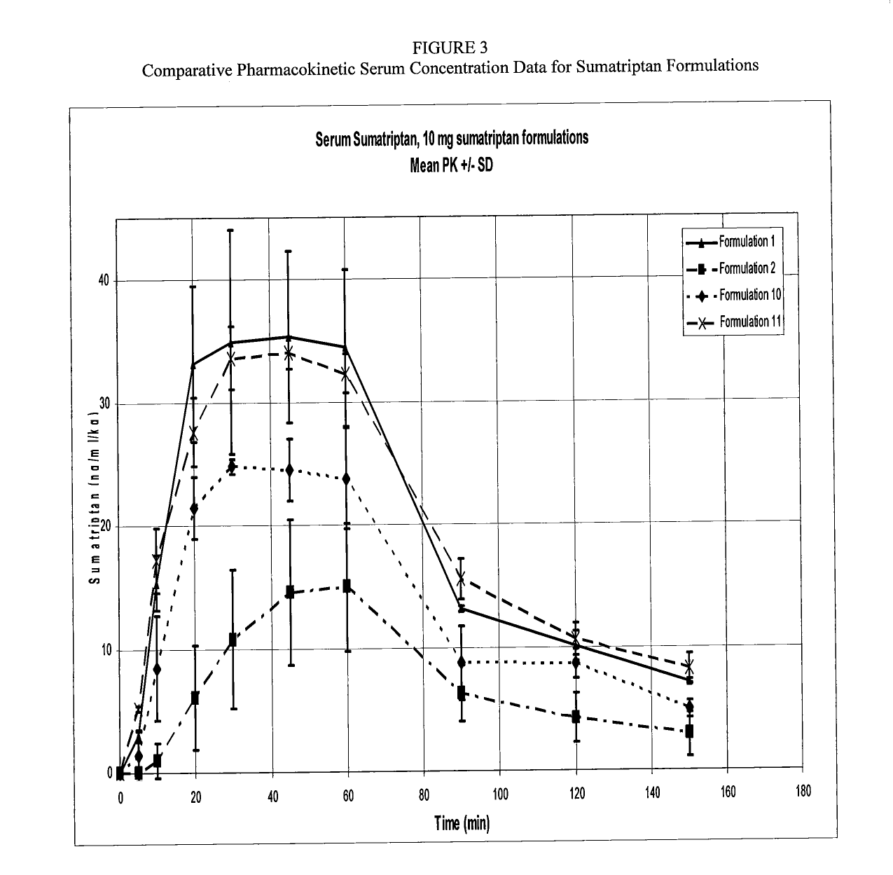

FIG. 3 shows the comparative pharmacokinetic data of powder blend formulations containing the primary ingredients sumatriptan succinate, sodium taurocholate and sodium chloride with varying adjusted osmolality values.

As can be seen from the above results, optimal sumatriptan absorption was observed with the formula having the 1000 mOs/kg osmolality conditions. Results also demonstrate that sumatriptan absorption in the presence of bile salt is a function of osmolality generated in the solution in contact with the buccal mucosa. However, sumatriptan absorption levels are relatively constant with osmolality values of about 1000 mOs/kg.

Additionally, increased toxicity appeared as edema at administration sites under the 2150 mOs/kg osmolality conditions. Because sumatriptan absorption through the canine buccal mucosa is not substantially increased at the 2150 mOs/kg experimental condition over that obtained at the 1000 mOs/kg condition, and because increased administration site irritation occurred at the 2150 mOs/kg condition, formulations that contain sodium taurocholate and generate at least 1000 mOs/kg appear to be optimal with regard to sumatriptan absorption.

### Example 4

### Comparative Powder Blends and Excipient Compatibility

A number of formulations were prepared and evaluated to determine excipient compatibilities, i.e., the effect on absorption when base constant formulations were prepared and combined with additional excipient ingredients. All of the formulations contained 14 mg sumatriptan succinate and 18 mg sodium taurocholate as powder blends, combined with different excipient ingredients (and control), then prepared 1.5 mL contact solutions with NaCl as the osmolality agent adjusted to 1000 mOs/kg for each formulation.

Formulations 1 and 2 were prepared in accordance with the procedure and composition set forth above. Formulation 12 was prepared by measuring 14 mg sumatriptan succinate, 18 mg sodium taurocholate, 45.5 sodium chloride and 30 mg glyceryl behenate (COMPRITOL® 888 ATO, available from Gattefosse Corp., Paramus, N.J.) into a small test tube and capping. The tube was then manually inverted several times to combine and mix the ingredients. The osmolality was 1000 mOs/kg (WESCOR® VAPRO® 5520) when measured in 1.5 g water.

Formulation 12

| Ingredient: | Amount (mg) | | Amount (% wt/wt) |
|---|---|---|---|
| Sumatriptan succinate | 14 | mg | 13% |
| Sodium taurocholate | 18 | mg | 17% |
| Sodium chloride | 45.5 | mg | 42% |
| Glyceryl behenate | 30 | mg | 28% |
| Total: | 107.5 | mg | 100% |

| | |
|---|---|
| Osmolality | 1000 mOs/kg (referenced to 1.5 g water) |

Formulation 13 was prepared by a procedure similar to that for Formulation 12, except 30 mg calcium phosphate dibasic was added (in place of glyceryl behenate) as a secondary ingredient. The osmolality was measured at 1000 mOs/kg in 1.5 g water.

Formulation 13

| Ingredient: | Amount (mg) | | Amount (% wt/wt) |
|---|---|---|---|
| Sumatriptan succinate | 14 | mg | 13% |
| Sodium taurocholate | 18 | mg | 17% |
| Sodium chloride | 45.5 | mg | 42% |
| Calcium phosphate dibasic | 30 | mg | 28% |
| Total: | 107.5 | mg | 100% |

| | |
|---|---|
| Osmolality | 1000 mOs/kg (referenced to 1.5 g water) |

Formulation 14 was prepared by a procedure similar to that for Formulation 12, except 30 mg magnesium stearate was used as a secondary ingredient. The osmolality was measured at 1000 mOs/kg in 1.5 g water.

Formulation 14

| Ingredient: | Amount (mg) | | Amount (% wt/wt) |
|---|---|---|---|
| Sumatriptan succinate | 14 | mg | 13% |
| Sodium taurocholate | 18 | mg | 17% |
| Sodium chloride | 45.5 | mg | 42% |
| Magnesium stearate | 30.0 | mg | 28% |
| Total: | 107.5 | mg | 100% |

| | |
|---|---|
| Osmolality | 1000 mOs/kg (referenced to 1.5 g water) |

Formulation 15 was prepared by a procedure similar to that for Formulation 12, except 35 mg crospovidone was added as a secondary ingredient. The osmolality was measured at 1000 mOs/kg in 1.5 g water.

Formulation 15

| Ingredient: | Amount (mg) | | Amount (% wt/wt) |
|---|---|---|---|
| Sumatriptan succinate | 14 | mg | 13% |
| Sodium taurocholate | 18 | mg | 16% |
| Sodium chloride | 45.5 | mg | 40% |
| Crospovidone | 35 | mg | 31% |
| Total: | 112.5 | mg | 100% |

| | |
|---|---|
| Osmolality | 1000 mOs/kg (referenced to 1.5 g water) |

Formulation 16 was prepared by a procedure similar to that for Formulation 12, except 35 mg silicified microcrystalline cellulose (PROSOLV® SMCC HD90) was added as a secondary ingredient. The osmolality was measured at 1000 mOs/kg in 1.5 g water.

Formulation 16

| Ingredient: | Amount (mg) | | Amount (% wt/wt) |
|---|---|---|---|
| Sumatriptan succinate | 14 | mg | 13% |
| Sodium taurocholate | 18 | mg | 16% |
| Sodium chloride | 45.5 | mg | 40% |
| Silicified microcrystalline | 35 | mg | 31% |
| cellulose | | | |
| Total: | 112.5 | mg | 100% |

| | |
|---|---|
| Osmolality | 1000 mOs/kg (referenced to 1.5 g water) |

The following table summarizes the formulations used in the experiment:

TABLE 4
Summary of Formulations 1, 2, 12 through 16

| | | | | | Osmolality |
|---|---|---|---|---|---|
| | | | | | (mOs/kg |
| | Sumatriptan | Sodium | Sodium | | measured in |
| Formulation: | succinate | taurocholate | chloride | Other | 1.5 g H₂O) |
| 1 (powder | 14 mg | 18 mg | 45.5 mg | | 1000 mOs/kg |
| blend) 100 mg | | | | | |
| 2 (powder | 14 mg | 18 mg | 14.5 mg | | 350 mOs/kg |
| blend) | | | | | |
| 100 mg | | | | | |
| 12 (powder | 14 mg | 18 mg | 45.5 mg | 30 mg Glyceryl | 1000 mOs/kg |
| blend) | | | | behenate | |
| 13 (powder | 14 mg | 18 mg | 45.5 mg | 30 mg calcium | 1000 mOs/kg |
| blend) | | | | phosphate dibasic | |
| 14 (powder | 14 mg | 18 mg | 45.5 mg | 30 mg magnesium | 1000 mOs/kg |
| blend) | | | | stearate | |
| 15 (powder | 14 mg | 18 mg | 45.5 mg | 35 mg | 1000 mOs/kg |
| blend) | | | | crospovidone | |
| 16 (powder | 14 mg | 18 mg | 45.5 mg | 35 mg silicified | 1000 mOs/kg |
| blend) | | | | microcrystalline | |
| | | | | cellulose | |

### In Vivo Canine Experiment

To develop dosage forms that utilize the invention, experiments were conducted to determine compatibility of various pharmaceutical ingredients typically used in compressed tablet dosage forms. The purpose of the experiment was to evaluate the possible effects of various secondary ingredients on transmucosal absorption of sumatriptan in compositions containing sumatriptan succinate, sodium taurocholate and sodium chloride. Except for Formulation 2, all formulation tested in this experiment had osmolality values of about 1000 mOs/kg.

Powder samples were introduced using the 1″ customized Teflon reservoir with the mucosa site hydrated with 1.5 mL deionized water and the hydration fluid discarded. The samples were initially dissolved in a test tube in 1.5 mL deionized water, and then transferred into the reservoir. The samples remained for a period of 60 minutes, followed by aspiration and rinsing of the reservoir/sampling site. The aspirated fluid was retained for analysis.

Blood samples (3.0 mL) were collected from the canine subjects at time intervals 0, 5, 10, 20, 30, 45, 60, 90, 120 and 150 minutes via indwelling catheter in cephalic vein. The samples were collected using VACUTAINER® serum separator and analyzed using mass spectroscopy.

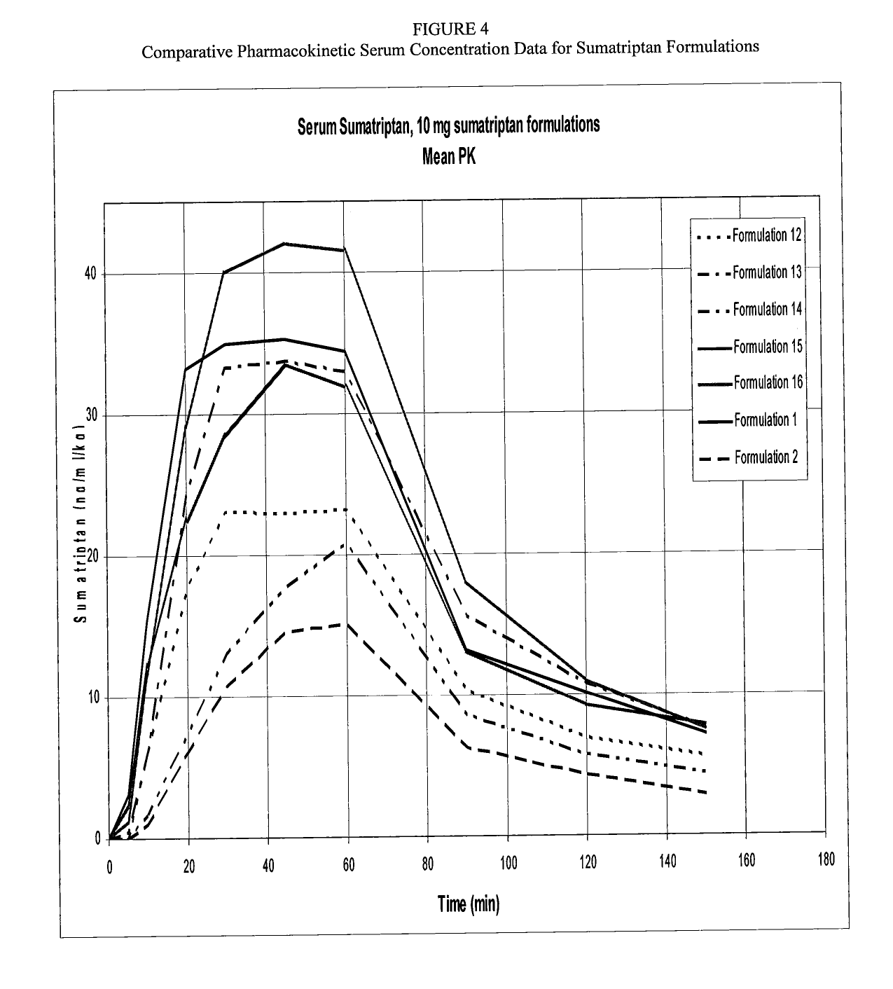

FIG. 4 shows comparative pharmacokinetic data in dogs using various formulations containing sumatriptan succinate, sodium taurocholate and sodium chloride in powder blends in combination with various secondary pharmaceutical ingredients, the compositions having osmolality levels of 1000 mOs/kg when measured in 1.5 mL water.

As can be seen from the data, specific ingredients such as magnesium stearate or glyceryl behenate appear to interfere with the absorption of sumatriptan and inhibit sumatriptan absorption across the mucosa. Other ingredients such as calcium phosphate dibasic, crospovidone, and silicified microcrystalline cellulose do not appear to have a significant inhibitory effect on the composition of the invention or sumatriptan absorption.

### Example 5

### Comparative Dosage Form Residence Times of Sumatriptan 10 mg Formulations

A compressed powder tablet referred to as Formulation 5 was prepared according to the invention containing sumatriptan succinate, sodium taurocholate, and sodium chloride as the osmolality adjusting ingredient from the powder blend set forth above in Formulation 1. Formulation 5 (Formulation 1 as compressed tablet) was evaluated in the anesthetized dog model to determine repeatability and consistency of sumatriptan absorption and delivery.

### In Vivo Canine Experiment

The in vivo experiment was conducted on the canine buccal mucosa using a procedure similar to that of the above experiments except that tablet residence time on the mucosa site was 10 minutes (as opposed to 60 minutes) and the amount of deionized water used was reduced to 0.6 mL. These shorter time and water conditions were selected to better represent available sublingual saliva amounts and intended residence time of the intended compressed powder tablet product.

The tablet samples were introduced to the dogs using the reservoir and 1.5 mL water pre-hydrated mucosa site for 10 minutes. The hydration fluid was aspirated and discarded, and 0.1 mL deionized water was deposited into the reservoir, immediately followed by deposition of the tablet sample into the reservoir onto the mucosa site and 0.10 mL deionized water. Once a minute, additional aliquots of 0.10 mL deionized water were added into the reservoir for a total of 5 minutes, totaling 0.60 mL deionized water added to the reservoir. The sample was maintained for 10 minutes, then aspirated from the reservoir and retained for analysis. The reservoir was twice rinsed with 1.5 mL deionized water and aspirate discarded. The reservoir was then removed and the site cleaned.

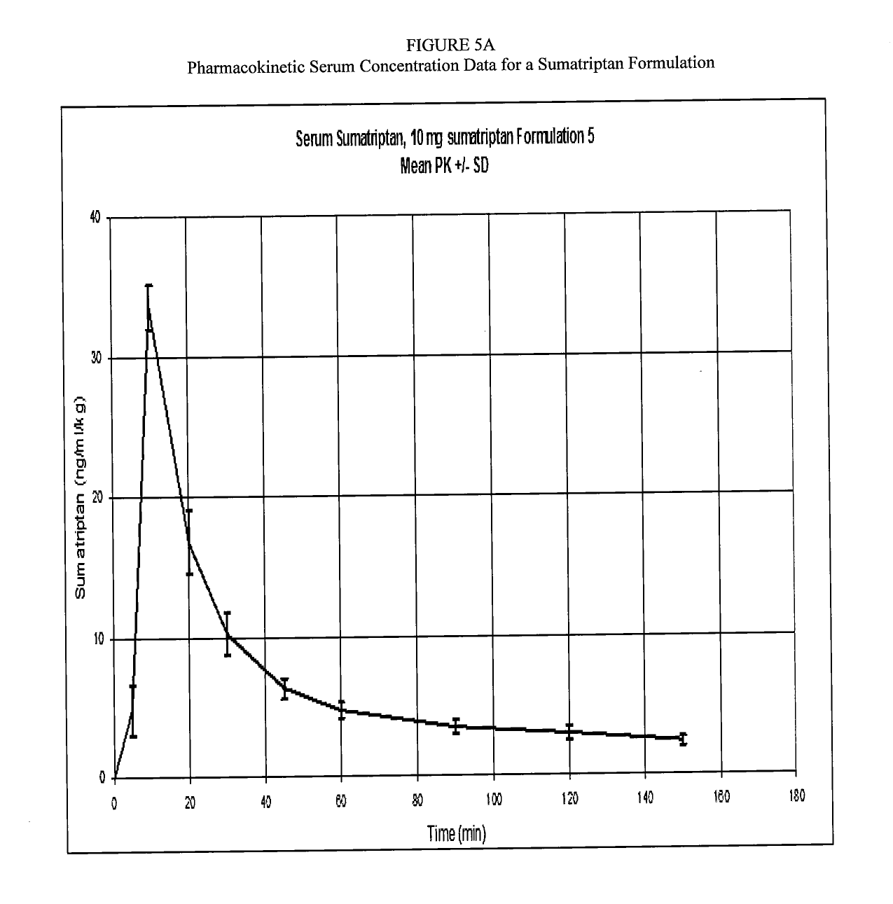

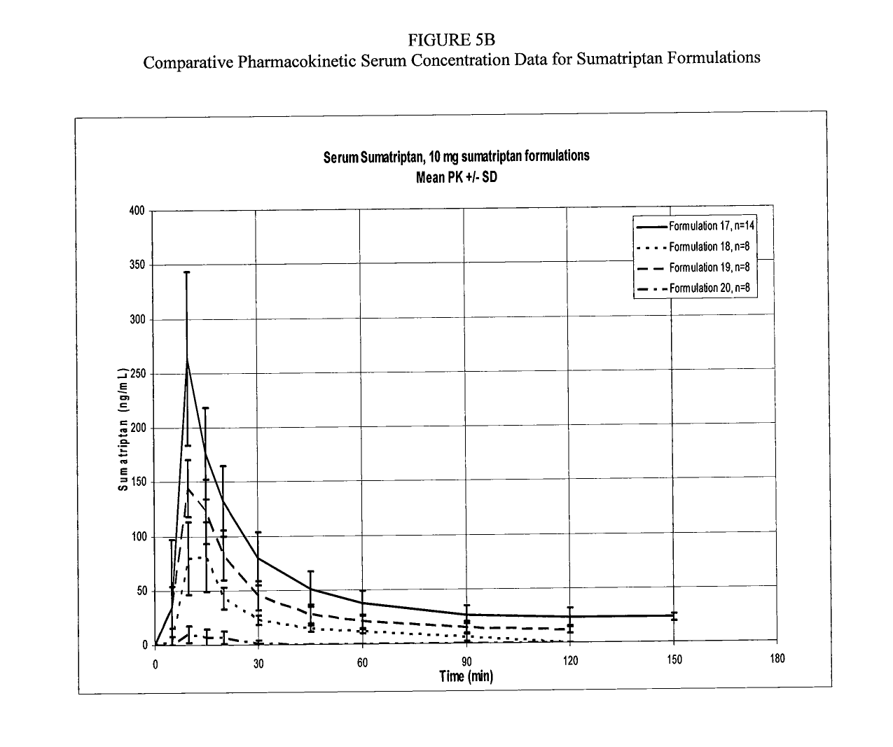

FIG. 5A shows pharmacokinetic data in dogs of sumatriptan formulations in compressed tablet form. The dosage form (compressed tablet) was delivered with a shorter residence time (10 minutes) as compared to the 60 minute exposure time as used for formulations in Example 1.

As can be seen from the data, rapid and extensive sumatriptan absorption across the canine buccal mucosa was achieved with Formulation 5 despite shorter tablet residence time of 10 minutes and despite smaller amounts of de-ionized water used to suspend the test tablet as compared to the formulations of Example 1. The difference in conditions was used to more closely mimic the anticipated oral conditions expected for the intended user of a tablet product. Based on the results, good reproducibility of sumatriptan absorption enhancement effect was observed using dosage forms prepared according to the invention.

### Example 6

### Preparation and Pharmacokinetic Data of Sumatriptan (10 mg) Formulations

Several different monolithic compressed powder sublingual tablets (round 5/16 inch diameter) were prepared having different formulations were prepared, each having a sumatriptan dosage of 10 mg (as sumatriptan succinate) and target tablet disintegration time of about 5 to 10 minutes following sublingual administration. The formulations differed with respect to combinations of sodium taurocholate content (varying between 10 mg and 20 mg) and adjusted osmolality (varying between 400 mOs/kg and 1000 mOs/kg at 1.5 mg reference), and corresponding total tablet weight/size. The dosage forms were then evaluated using the anesthetized dog model to determine optimal ranges of ingredients sodium taurocholate and sodium chloride, and their effects on sumatriptan absorption.

Individual ingredients were dispensed in sufficient quantity to achieve the quantity of powder blend with a composition weight/weight basis as give in Table 5 (Formulation 17), Table 6 (Formulation 18), Table 7 (Formulation 19) and Table 8 (Formulation 20).

The formulations were generally prepared as follows. Sodium chloride, maltodextrin (if in the composition), potassium phosphate, sumatriptan succinate, sodium taurocholate, sucralose, sodium bicarbonate, and microcrystalline cellulose (if in the composition) were transferred to a suitable container and blended for a period of about 50 minutes at 15 rpm in a TURBULA® T10-B apparatus. Then, magnesium stearate was added to the container, and the mixture blended for an additional 10 minutes at 15 rpm. The resulting powder blend was then transferred to a rotary tablet press (PICCOLA® Type-D) and compressed to form flat-faced, beveled-edge (FFBE) 5/16″ tablets with a target weight and compression force as indicated in the following tables. The resulting formulations are set forth in the following table:

TABLE 5
Formulation 17 ( 5/16″ Diameter FFBE 110 mg Weight Tablets)

| | 17a | 17b | 17c | 17d | 17e |
|---|---|---|---|---|---|
| | Amount | Amount | Amount | Amount | Amount |
| Ingredient | (wt %) | (wt %) | (wt %) | (wt %) | (wt %) |
| Sumatriptan succinate | 14.0 | 12.7 | 12.7 | 12.7 | 12.7 |
| Sodium taurocholate | 18.2 | 18.2 | 18.2 | 18.2 | 18.2 |
| Sodium chloride | 25.7 | 32.2 | 32.2 | 32.2 | 32.2 |
| Potassium phosphate | 35.0 | 18.2 | 18.2 | 18.2 | 18.2 |
| monobasic | | | | | |
| Sodium bicarbonate | 5.00 | 4.55 | 4.55 | 4.55 | 4.55 |
| Microcrystalline | 0 | 12.3 | 12.3 | 11.9 | 11.9 |
| cellulose | | | | | |
| Magnesium stearate | 1.50 | 1.25 | 1.25 | 1.25 | 1.25 |
| Sucralose | 0.60 | 0.60 | 0.60 | 1.00 | 1.00 |
| Total (mg) | | | | | |
| Compression force (kN) | 1.1 ± 0.1 | 3.2 ± 0.4 | 2.9 ± 0.2 | 3.5 ± 0.4 | 4.2-4.3 |
| Batch size (g) | 100 | 80 | 80 | 500 | 500 |

TABLE 6
Formulation 18 ( 5/16″ diameter FFBE 100 mg Tablet Weight)

| | 18a | 18b | 18c | 18d |
|---|---|---|---|---|
| | Amount | Amount | Amount | Amount |
| Ingredient | (wt %) | (wt %) | (wt %) | (wt %) |
| Sumatriptan succinate | 14.0 | 14.0 | 14.0 | 14.0 |
| Sodium taurocholate | 20.0 | 20.0 | 20.0 | 20.0 |
| Sodium chloride | 4.91 | 4.76 | 4.76 | 4.76 |
| Potassium phosphate | 20.0 | 20.0 | 20.0 | 20.0 |
| monobasic | | | | |
| Sodium bicarbonate | 5.0 | 5.0 | 5.0 | 5.0 |
| Microcrystalline | 34.5 | 24.0 | 24.0 | 24.0 |
| cellulose | | | | |
| Magnesium stearate | 1.00 | 1.25 | 1.25 | 1.25 |
| Sucralose | 0.60 | 1.00 | 1.00 | 1.00 |
| Maltodextrin | 0 | 10.0 | 10.0 | 10.0 |
| Total (mg) | | | | |
| Compression force (kN) | 3.8 ± 0.3 | 3.6 ± 0.4 | 4.9 ± 0.6 | 4.2 to 4.6 |
| Batch size (g) | 80 | 80 | 500 | 500 |

TABLE 7
Formulation 19 ( 5/16″ diameter, FFBE, 100 mg Tablet Weight

| | 19a | 19b | 19c |
|---|---|---|---|
| | Amount | Amount | Amount |
| Ingredient | (wt %) | (wt %) | (wt %) |
| Sumatriptan succinate | 14.0 | 14.0 | 14.0 |
| Sodium taurocholate | 10.0 | 10.0 | 10.0 |
| Sodium chloride | 33.8 | 35.8 | 35.8 |
| Potassium phosphate | 20.0 | 20.0 | 20.0 |
| monobasic | | | |
| Sodium bicarbonate | 5.00 | 5.00 | 5.00 |
| Microcrystalline | 5.00 | 8.00 | 8.00 |
| cellulose | | | |
| Magnesium stearate | 1.25 | 1.25 | 1.25 |
| Sucralose | 1.00 | 1.00 | 1.00 |
| Maltodextrin | 10.0 | 5.0 | 5.0 |
| Total (mg) | | | |
| Compression force (kN) | 4.6 ± 0.4 | 5.5 ± 0.5 | 5.0 to 5.7 |
| Batch size (g) | 80 | 80 | 500 |

TABLE 8
Formulation 20 5/16″ diameter FFBE 90 mg Tablet Weight

| | 20a | 20b | 20c | 20d |
|---|---|---|---|---|
| | Amount | Amount | Amount | Amount |
| Ingredient | (wt %) | (wt %) | (wt %) | (wt %) |
| Sumatriptan succinate | 15.6 | 15.6 | 15.6 | 15.6 |
| Sodium taurocholate | 11.1 | 11.1 | 11.1 | 11.1 |
| Sodium chloride | 5.81 | 5.81 | 5.81 | 5.81 |
| Potassium phosphate | 22.2 | 22.2 | 20.0 | 22.2 |
| monobasic | | | | |
| Sodium bicarbonate | 5.56 | 5.56 | 5.56 | 5.56 |
| Microcrystalline | 17.5 | 17.5 | 17.5 | 17.5 |
| cellulose | | | | |
| Magnesium stearate | 1.25 | 1.25 | 1.25 | 1.25 |
| Sucralose | 1.00 | 1.00 | 1.00 | 1.00 |
| Maltodextrin | 20.0 | 30.0 | 22.2 | 20.0 |
| Total (mg) | | | | |
| Compression force (kN) | 6.3 ± 0.6 | 5.4 ± 0.5 | 6.2 ± 0.8 | 6.3 to 6.7 |
| Batch size (g) | 80 | 80 | 500 | 500 |

FIG. 5B shows comparative pharmacokinetic serum concentration data for sumatriptan formulations. In FIG. 5B, each curve is the collective average of the serum values obtained from each sub-formula. For example, Formulation 19 comprises three sub-formulations 19a, 19b, and 19c. Each sub-formula 19a, 19b, and 19c was evaluated in at least two dogs and the curve for Formulation 19 in FIG. 5B is the average of the serum results from 8 dogs with the idea that the formulations were close enough in compositions and the PK results similar enough to group the results. The curve for Formulation 17 is the average of the serum results for five sub-formulas studied in 14 dogs. The curve for Formulation 18 is the average of the serum results for four sub-formulas studied in 8 dogs. The curve for Formulation 20 is the average of the serum results for four sub-formulas studied in 8 dogs.

As can be seen from the data graphed in FIG. 5B, serum sumatriptan levels are substantially affected by the amount of sodium taurocholate and osmolality adjusting ingredients. In addition to other ingredients, Formulations 17 and 18 incorporate 20 mg sodium taurocholate and osmolality adjusting ingredients sufficient to achieve an osmolality of about 1000 and about 400 mOs/kg when measured in 1.5 g water, respectively. In addition to other ingredients, Formulations 19 and 20 incorporate 10 mg sodium taurocholate and osmolality adjusting ingredient sufficient to achieve osmolality levels of about 1000 and about 400 mOs/kg, respectively. The results also indicate that the amount of osmolality adjusting ingredients may have a more substantial effect on sumatriptan absorption across the canine buccal mucosa than the amount sodium taurocholate. However, as shown in FIGS. 1 and 2, bile salts in general and sodium taurocholate in particular, must be present in the composition to achieve the absorption enhancement effect of the hyperosmotic condition.

### Example 7

### Comparative Pharmacokinetic Study with Zolmitriptan

In an effort to determine the dose response of zolmitriptan in compositions containing sodium taurocholate at conditions that generate about 1000 mOs/kg on zolmitriptan absorption, different formulations were prepared and administered using the anaesthetized dog models.

Formulation 21 Preparation of Effervescent Tablet with 2000 mOs/kg (Measured in 1.5 g Water)

A 100 g powder blend was prepared by first combining zolmitriptan and half of the total mannitol into a suitable container and manually mixed by inverting the container several times. Next, sodium bicarbonate, sodium carbonate, citric acid, the remaining mannitol, sodium starch glycolate were transferred into the container and blended on a low shear tumble mixer (TUBULA® T10-B) for a period of about 60 minutes at 15 rpm. Magnesium stearate was then added and blended for an additional 10 minutes at 15 rpm. Individual ingredients were dispensed in sufficient quantity to achieve a 100 g powder blend with a composition on a weight/weight basis of 48.5% mannitol, 21% sodium bicarbonate, 15% citric acid, 10% anhydrous sodium carbonate, 3% sodium starch glycolate, 2% magnesium stearate and 0.5% zolmitriptan.

The powder blend was transferred to a rotary tablet press (Piccola Type-D) and compressed into flat-faced beveled edge 5/16″ tablets with a target weight of 200 mg. The resulting formulation achieves an osmolality of about 1000 mOs/kg (Wescor Vapro 5520) when measured in 1.5 g water at compression force 5.4 kN.

Formulation 21

| Ingredient: | Amount (mg) | | Amount (% wt/wt/) |
|---|---|---|---|
| Zolmitriptan (free base) | 1.0 | mg | 0.5% |
| Sodium bicarbonate | 42.0 | mg | 21.0% |
| Citric acid | 30.0 | mg | 15.0% |
| Sodium carbonate (anhydrous) | 20.0 | mg | 10.0% |
| Mannitol | 97.0 | mg | 48.5% |
| Sodium starch glycolate | 6.0 | mg | 3.0% |
| Magnesium stearate | 4.0 | mg | 2.0% |
| Total: | 200 | mg | 100.0% |

| | |
|---|---|
| Osmolality | 1000 mOs/kg (referenced to 1.5 g water) |

Formulation 22 was prepared by measuring 1 mg zolmitriptan and 10 mg sodium taurocholate into a small test tube. The tube was capped and manually inverted several times to blend the components.

Formulation 22 Non effervescent Zolmitriptan Formulation

| Ingredient: | Amount (mg) | Amount (% wt/wt) |
|---|---|---|
| Zolmitriptan | 1 mg | 9% |
| Sodium taurocholate | 10 mg | 91% |
| Total: | 11 mg | 100% |

### In Vivo Canine Experiment

The study was approved by IACUC and performed under protocol. The experimental objective was to evaluate the effects of osmolality on a compressed powder tablet composition containing zolmitriptan and other ingredients of an effervescent formulation on the absorption of zolmitriptan through canine buccal mucosa. The effervescent test sample does not contain sodium taurocholate. Anesthetized male and female purpose bred Beagle dogs were used weighing between 8 and 12 kg.

Tablets were introduced to the dogs using a custom-designed 1″ Teflon reservoir adhered to the mucosa. The site is hydrated with 1.5 mL de-ionized water for 10 minutes and aspirated and discarded. The tablet is deposited into the reservoir and followed by 0.75 mL deionized water. The sample is maintained for a period of about 60 minutes exposure time, aspirated and retained for analysis. The reservoir is twice rinsed with 1.5 mL deionized water, aspirated and discarded. The site is finally cleaned.

Blood samples (3 mL) were collected via indwelling catheter placed in the cephalic vein at time intervals 0, 5, 10, 20, 30, 45, 60, 90, 120 and 180 minutes post-dose using serum separator VACUTAINER® The samples were analyzed by mass spectroscopy using methods and techniques known in the art.

Using a procedure and models similar to that above, the objective was to evaluate the effects of osmolality on powder compositions containing zolmitriptan and sodium taurocholate on the absorption of zolmitriptan through canine buccal mucosa.

Powder samples of Formulation 22 were introduced into the 10-minute pre-hydrated reservoir. Immediately, the sample was hydrated after deposit with 0.75 mL phosphate buffered saline instead of water to achieve an osmolality of 320 or 1000 mOs/kg in the reservoir. The sample was maintained for a period of about 60 minutes, and then aspirated and retained for analysis. The reservoir was twice rinsed with 1.5 mL deionized water and discarded. The reservoir is removed and cleaned.

The samples (3 ml) were collected and analyzed using techniques and equipment similar to that used for the testing of Formulation 21.

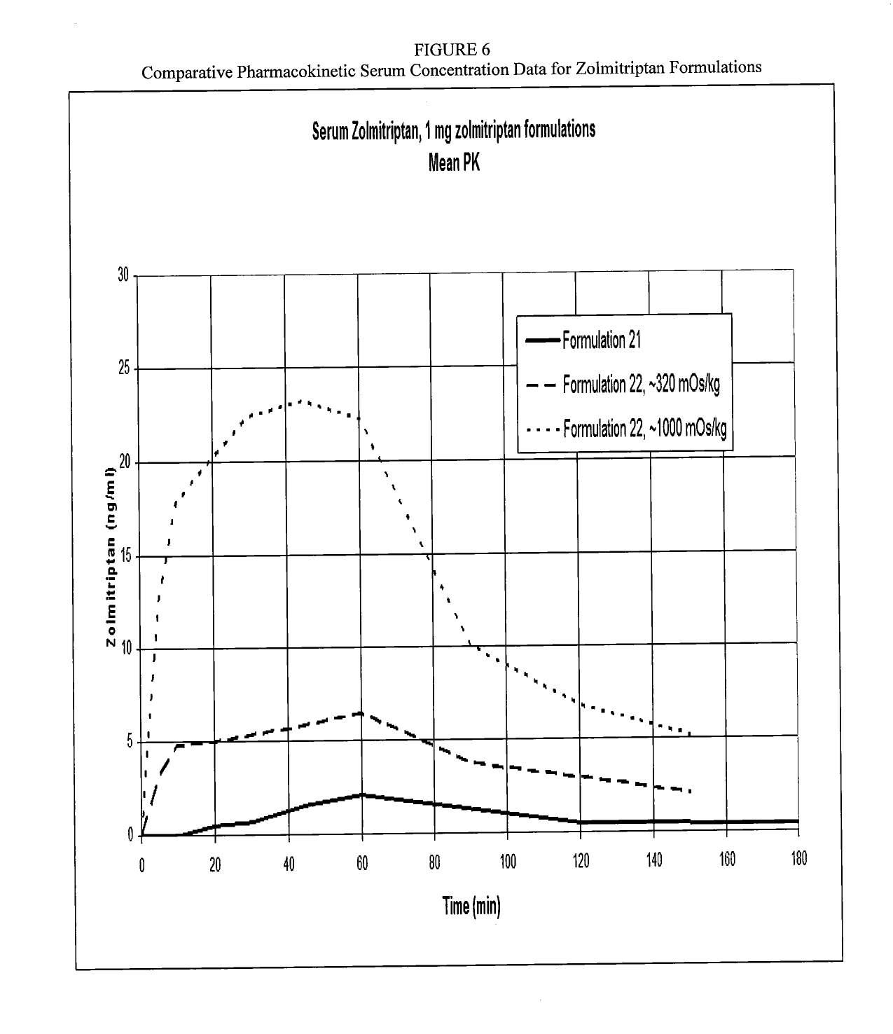

FIG. 6 shows comparative pharmacokinetic data of various formulations in canine subjects, but with the active zolmitriptan (free base form) instead of sumatripan in both compressed tablets and powder blends. The tablets were effervescent dosage forms with osmolalities of about 1000 mOs/kg (when measured in 1.5 g water) but absent sodium taurocholate. The powder formulations contained zolmitriptan and sodium taurocholate in phosphate buffered saline solution that generates either 320 or 1000 mOs/kg.

As can be seen from the above results, an increase in zolmitriptan was observed as the osmolality of the composition increased from 320 to 1000 mOs/kg osmolality conditions. The results also show that zolmitriptan absorption in the presence of a given amount of sodium taurocholate is a function of osmolality generated in the solution in contact with the buccal mucosa. However, little zolmitriptan absorption was observed in the composition absent sodium taurocholate even though the osmolality of the composition was equivalent to the composition containing sodium taurocholate.

### Example 8

### Comparative Powder Blend Formulations with Varying Osmolalities

In an effort to determine the effect of ranges of osmolality on zolmitriptan absorption, different formulations were prepared and administered using the canine models.

Formulation 23 was prepared by initially measuring 1 mg zolmitriptan, 10 mg sodium taurocholate, and 16 mg sodium chloride into a small test tube. The test tube was capped and manually inverted several times to blend the components. Osmolality was measured at 1000 mOs/kg (WESCOR®VAPRO® 5520) when measured in 0.75 mL pH 6 citrate/phosphate buffer (62% of 0.2 M Na₂HPO₄.7H2O and 38% 0.1 M C₆H₈O₇).

Formulation 23

| Ingredient: | Amount (mg) | Amount (% wt/wt) |
|---|---|---|
| Zolmitriptan | 1 mg | 4% |
| Sodium taurocholate | 10 mg | 37% |
| Sodium chloride | 16 mg | 59% |
| Total: | 27 mg | 100% |

| | |
|---|---|
| Osmolality | 1000 mOs/kg (0.75 ml citrate/phosphate buffer) |

Formulation 24 was a powder blend containing sodium taurocholate prepared by initially combining 2.5 mg zolmitriptan, 11.7 mg sodium taurocholate and 15.9 mg sodium chloride into a small test tube. The test tube was capped and manually inverted several times to mix the ingredients. Osmolality was 1000 mOs/kg (WESCOR®VAPRO® 5520) when measured in 0.75 mL pH 6 citrate/phosphate buffer (62% of 0.2 M Na₂HPO₄.7H2O and 38% 0.1 M C₆H₈O₇).

Formulation 24

| Ingredient: | Amount (mg) | Amount (% wt/wt) |
|---|---|---|
| Zolmitriptan | 2.5 mg | 8% |
| Sodium taurocholate | 11.7 mg | 39% |
| Sodium chloride | 15.9 mg | 53% |
| Total: | 30.1 mg | 100% |

| | |
|---|---|
| Osmolality | 1000 mOs/kg (0.75 ml, pH 6 citrate/ |
| | phosphate buffer) |

### In Vivo Canine Experiment

The objective of the experiment was to evaluate the zolmitriptan dose response in formations containing sodium taurocholate and generate 1000 mOs/kg on the absorption of zolmitriptan through canine buccal mucosa.

Anesthetized canine subjects were male and female Beagles between 8 and 12 kg. Powder samples were introduced to the dogs using a custom 1″ diameter reservoir adhered to the buccal mucosa. The site was hydrated for 10 minutes with 1.5 mL deionized water, and then the fluid aspirated and discarded. The powder test samples were then introduced directly onto the mucosa, followed immediately by 0.75 mL pH 6 citrate/phosphate buffer and allowed to reside for a period of 60 minutes. The reservoir sample is then aspirated and retained for analysis, and the site twice rinsed with 1.5 mL deionized water. The reservoir is removed and the site cleaned.

Blood samples (3.0 mL1) were collected via indwelling catheter in cephalic vein at time intervals 0, 5, 10, 20, 30, 45, 60, 90, 120 and 150 minutes post-dose using serum separator VACUTAINER®. The samples were then analyzed using mass spectroscopy using readily known techniques.

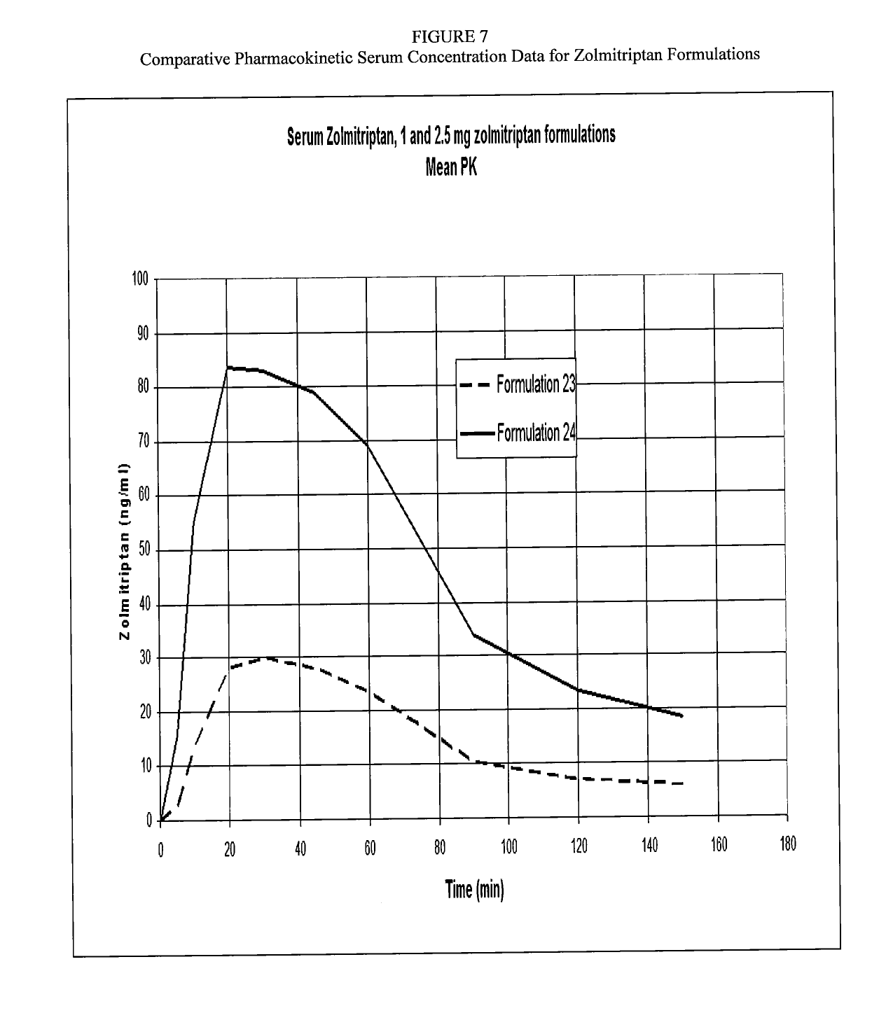

FIG. 7 shows comparative pharmacokinetic data of various formulations in canine subjects containing zolmitriptan (free base) in powder blends containing sodium taurocholate and two levels of zolmitriptan dosing. Osmolality was adjusted in the powder blends with sodium chloride.

As can be seen from the above data, increasing serum zolmitriptan were observed with increasing zolmitriptan dose. The results show that zolmitriptan absorption increases with increased zolmitriptan dose, indicating that the same general absorption enhancement mechanism was present for a given amount of sodium taurocholate and osmolality at the two dosage amounts.

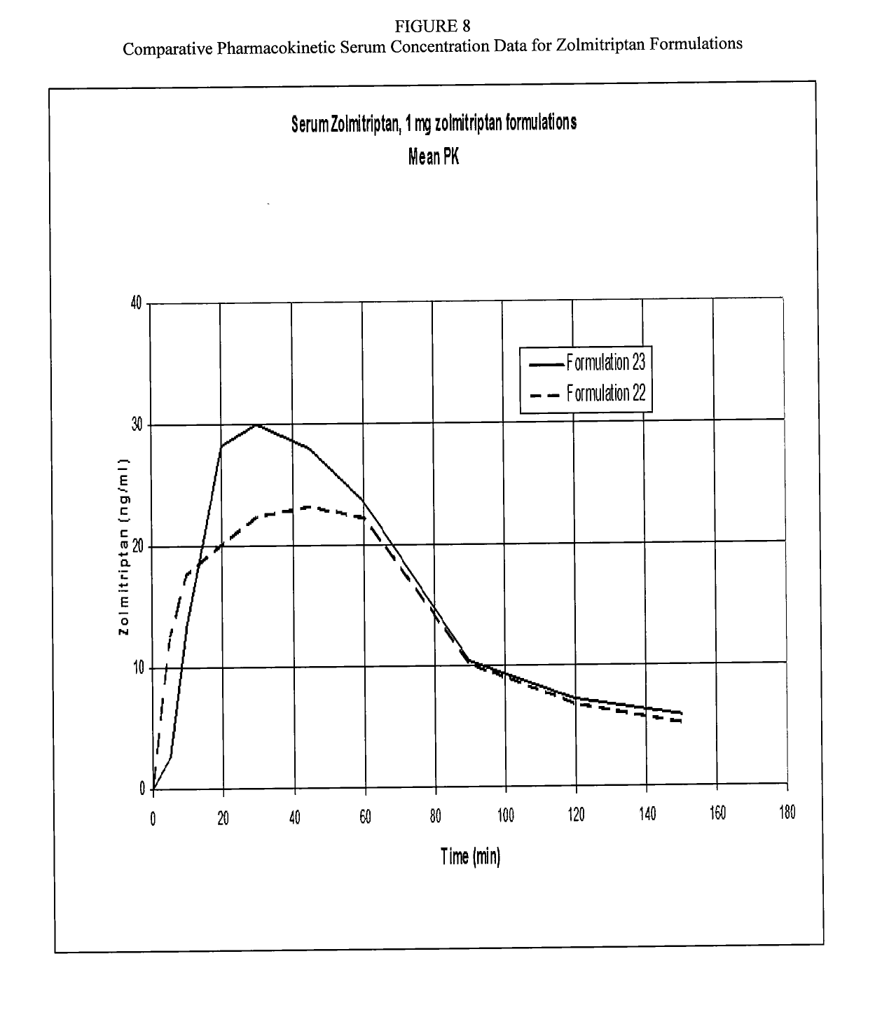

FIG. 8 shows comparative pharmacokinetic data of various formulations in canine subjects containing zolmitriptan and sodium taurocholate in powder blends. In one experiment, osmolality is adjusted using phosphate buffered saline, while the other osmolality is adjusted using sodium chloride incorporated into the powder blend.

As can be seen from the results, serum zolmitriptan levels were approximately equal whether osmolality was provided by phosphate buffered saline used in the experiment or by sodium chloride added to the powder blend. The results show that zolmitriptan absorption is independent of the source of osmolality adjusting ingredient at these two conditions. However, zolmitriptan absorption does appear to be faster in the experimental condition using phosphate buffered saline. This can be attributed to the fact that the phosphate buffered solution immediately provided the 1000 mOs/kg osmolality for absorption enhancement while the sodium chloride as a dry powdered ingredient must first dissolve into solution before generating the osmolality condition.

### INDUSTRIAL APPLICABILITY

The invention is useful in the preparation of pharmaceutical compositions and dosage forms wherein the desired drug delivery route is the oral transmucosal route and the onset is relatively rapid (e.g., the Cₘₐₓ occurs at a relatively short tₘₐₓ). The invention is particularly useful for the delivery of indole compounds, such as triptans, some of which are known to be useful in treating migraine headaches wherein rapid onset provides desirable fast relief effects.

The invention has been described herein above with reference to various and specific embodiments and techniques. It will be understood by one skilled in the art, however, that reasonable modifications and variations can be made from such embodiments and techniques without substantially departing from either the spirit or scope of the invention as defined by the claims.

What is claimed is:

1. A transmucosal pharmaceutical composition comprising:
a) an active compound;
b) a bile salt; and
c) an osmolality adjusting ingredient;
wherein said osmolality adjusting ingredient generates a localized hyperosmotic environment and maintains an osmolality level for a period of time sufficient to produce hypertonicity-facilitated transmucosal transport of said active compound across the mucosal tissue.

2. The composition according to claim 1, wherein said active compound is a triptan.

3. The composition according to claim 2, wherein said triptan is selected from the group consisting of sumatriptan and zolmitriptan.

4. The composition according to claim 1, wherein said bile salt is sodium taurocholate.

5. The composition according to claim 1, wherein said osmolality adjusting ingredient comprises sodium chloride.

6. The composition according to claim 1, wherein said osmolality adjusting ingredient generates a hyperosmotic condition and osmolality ranging from about 400 mOs/kg to about 2000 mOs/kg as measured in 1.5 g water.

7. The composition according to claim 6, wherein said osmolality ranges from about 800 mOs/kg to about 1000 mOs/kg as measured in 1.5 g water.

8. A solid transmucosal dosage form comprising:
a. an active compound;
b. a bile salt;
c. an osmolality adjusting ingredient;
wherein said osmolality adjusting ingredient generates a localized hyperosmotic environment maintains an osmolality level for a period of time sufficient to produce hypertonicity-facilitated transmucosal transport of said active compound across the mucosal tissue.

9. The dosage form according to claim 8, wherein said dosage form is in the form of an oral transmucosal tablet formulated for resident placement within a recipient's oral cavity for transmucosal delivery of said active compound across said recipient's oral mucosal tissue.

10. The dosage form according to claim 8, wherein said active compound is a triptan.

11. The dosage form according to claim 10, wherein said triptan is selected from the group consisting of sumatriptan and zolmitriptan.

12. The dosage form according to claim 8, wherein said bile salt is sodium taurocholate.

13. The dosage form according to claim 8, wherein said osmolality adjusting ingredient is sodium chloride.

14. The dosage form according to claim 8, wherein said osmolality adjusting ingredient generates a hyperosmotic environment and osmolality ranging from about 400 mOs/kg to about 2000 mOs/kg as measured in 1.5 g water.

15. The dosage form according to claim 14, wherein said osmolality ranges from about 800 mOs/kg to about 1000 mOs/kg as measured in 1.5 g water.

16. A method of treating migraine headaches in a recipient comprising administering to said recipient in need of said treatment a therapeutically effective amount of a transmucosal pharmaceutical composition in dosage form, said dosage form comprising:
a triptan compound;
a bile salt; and
an osmolality adjusting ingredient;
wherein said osmolality adjusting ingredient generates a localized hyperosmotic environment and maintains an osmolality level for a period of time sufficient to produce hypertonicity-facilitated transmucosal transport of said active compound across the mucosal tissue.

17. The method according to claim 16, comprising the steps of:
a) providing to said recipient said dosage form;
b) placing said dosage form in the recipient's oral cavity; and
c) permitting dosage form to reside in situ and disintegrate in situ for a period of time sufficient to permit transmucosal delivery of a therapeutically effective amount of the active compound across the recipient's mucosa.

18. The method according to claim 16, wherein said triptan compound is selected from the group consisting of sumatriptan and zolmitriptan.

19. The method according to claim 16, wherein said bile salt is sodium taurocholate.

20. The method according to claim 16, wherein said osmolality adjusting ingredient is sodium chloride.

21. The method according to claim 16, wherein said dosage form generates hyperosmotic environment and osmolality ranging from about 400 mOs/kg to about 2000 mOs/kg as measured in 1.5 g water.

22. The method according to claim 19, wherein said osmolality ranges from about 800 mOs/kg to about 1000 mOs/kg as measured in 1.5 g water.

23. A method of enhancing oral transmucosal absorption of an active compound in a mammal comprising:
a) preparing a composition comprising a combination of active compound together with a bile salt; and
b) combining the ingredients of step a) with an osmolality adjusting ingredient;
wherein the combination of ingredients of step a) and step b), in any order, creates osmolality levels consistent with hypertonic conditions and facilitate transport of said active compound across mucosa when said ingredients are placed in situ adjacent oral mucosal tissue of said mammal.

24. The method according to claim 23, wherein said active compound is a triptan.

25. The method according to claim 24, wherein said triptan is selected from the group consisting of sumatriptan and zolmitriptan.

26. The method according to claim 23, wherein said bile salt is sodium taurocholate.

27. The method according to claim 23, wherein said osmolality adjusting ingredient is sodium chloride.

28. The method according to claim 16, wherein said dosage form generates hyperosmotic environment and osmolality ranging from about 400 mOs/kg to about 2000 mOs/kg as measured in 1.5 g water.

29. The method according to claim 19, wherein said osmolality ranges from about 800 mOs/kg to about 1000 mOs/kg as measured in 1.5 g water.

30. A transmucosal pharmaceutical composition comprising:
a) an active compound;
b) a bile salt in an amount of from about 5% to about 30% by weight of the composition; and
c) an osmolality adjusting ingredient present in an amount that results in an osmolality of between about 400 mOs/kg to about 2000 mOs/kg when the osmolality of the transmucosal pharmaceutical composition is measured in 1.5 g of water.

31. The transmucosal pharmaceutical composition of claim 30, wherein the active compound is present in an amount of from about 5% to about 25% by weight of the composition.

32. The transmucosal pharmaceutical composition of claim 30, wherein the bile salt is present in an amount of from about 5% to about 30% by weight of the composition.

33. The transmucosal pharmaceutical composition of claim 30, wherein the bile salt is present in an amount of from about 10% to about 20% by weight of the composition.

34. The transmucosal pharmaceutical composition of claim 30, wherein the osmolality adjusting ingredient is present in an amount that results in an osmolality of between about 800 mOs/kg to about 1000 mOs/kg when measured in 1.5 g of water.

35. The transmucosal pharmaceutical composition of claim 30, wherein the active compound is a triptan compound or a salt of a triptan compound.

36. The transmucosal pharmaceutical composition of claim 35, wherein the triptan compound or salt of a triptan compound is selected from: rizatriptan benzoate, naratriptan hydrochloride, frovatriptan succinate, eletriptan hydrobromide, almotriptan malate, sumatriptan succinate, and zolmitriptan.

37. The transmucosal pharmaceutical composition of claim 30, wherein the bile salt is selected from a taurocholate salt, a glycocholate salt, a glycodeoxycholate salt, a taurodeoxycholate salt, a cholate salt, a taurochenodeoxycholate salt, a tauroursodeoxycholate salt, and combinations thereof.

38. The transmucosal pharmaceutical composition of claim 37, wherein the bile salt is a sodium salt.

39. The transmucosal pharmaceutical composition of claim 37, wherein the bile salt is sodium taurocholate.

40. The transmucosal pharmaceutical composition of claim 30, wherein the osmolality adjusting ingredient is selected from potassium chloride, calcium chloride, sodium lactate, sodium chloride, dextrose, mannitol, sucrose, trehalose, and phosphate buffered saline, and mixtures thereof.

41. The transmucosal pharmaceutical composition of claim 40, wherein the osmolality adjusting ingredient is sodium chloride.

42. The transmucosal pharmaceutical composition of claim 30 in solid dosage form.

43. A method of treating or alleviating one or more symptoms associated with a migraine headache in a subject comprising administering to the subject a therapeutically effective amount of the transmucosal pharmaceutical composition of claim 35.

44. A transmucosal pharmaceutical composition comprising:
a) a triptan compound or salt thereof;
b) sodium taurocholate in an amount of from about 10% to about 20% by weight of the composition; and
c) sodium chloride in an amount that results in an osmolality of the composition of between about 800 mOs/kg to about 1000 mOs/kg when measured in 1.5 g of water.

45. A method of treating or alleviating one or more symptoms associated with a migraine headache in a subject comprising administering to the subject a therapeutically effective amount of the transmucosal pharmaceutical composition of claim 44.
